(19) United States
(12) Patent Application Publication
(10) Pub. No.: US 2024/0408044 A1
(43) Pub. Date: Dec. 12, 2024

(54) COMPOSITION

(71) Applicant: SOCIETE DES PRODUITS NESTLE S.A., Vevey (CH)

(72) Inventors: CLAUDIA ROESSLE, Morges (CH); DENIS BREUILLE, Lausanne (CH); MICKAEL HARTWEG, Carrouge (CH)

(21) Appl. No.: 18/703,117

(22) PCT Filed: Oct. 17, 2022
(86) PCT No.: PCT/EP2022/078765

§ 371 Date: Apr. 19, 2024

(30) Foreign Application Priority Data
Oct. 21, 2021 (EP) 21203852.5

(51) Int. Cl. A61K 31/198; A61P 21/00

(52) U.S. Cl. CPC A61K 31/198; A61P 21/00

(57) ABSTRACT

The present invention relates to an amino acid composition comprising threonine, serine, proline, leucine, and cysteine for use in preventing and/or reducing glutamine deficiency and/or arginine deficiency in a subject. The present invention also relates to an enteral nutritional composition comprising threonine, serine, proline, leucine, and cysteine and a method for producing the same.

### FIELD OF THE INVENTION

The present invention relates to an amino acid composition for use in preventing and/or reducing glutamine deficiency and/or arginine deficiency in a subject. The present invention also relates to an enteral nutritional composition and a method for producing the same.

### BACKGROUND TO THE INVENTION

Systemic inflammation in critically ill patients can lead to significant anabolism in the splanchnic area, especially in the gut and the liver. During sepsis, dietary requirements of cysteine for glutathione synthesis are doubled (Malmezat T, et al. J Nutr 2000; 130 (5): 1239-1246). Amino acids are also implicated in the repair of the intestinal mucosa as well as in providing energy to bolster the innate defence of the gut (Su L, et al. PloS One 2015; 10 (4): e0121933; and Jespersen J G, et al. PLOS One 2011; 6 (3): e18090).

The large utilisation of amino acids by the gut and the liver induce a significant increase of dietary requirements. Several nonessential amino acids may become essential under such conditions. Arginine and glutamine are two such conditionally essential amino acids. Glutamine, an arginine precursor, is one of the most abundant amino acids in the body and can become deficient in critically ill patients (Morris, C. R., et al., 2017. Nutrition in Clinical Practice, 32, pp. 30S-47S). Arginine deficiency in critical illness is associated with impairments in microcirculatory blood flow, impaired wound healing, and T-cell dysfunction (Patel, J. J., et al., 2016. Nutrition in Clinical Practice, 31 (4), pp. 438-444).

In addition to amino acid deficiency, most patients in intensive care units (ICUs) experience muscle wasting (Kang, M. C., 2020. Surgical Metabolism and Nutrition, 11 (2), pp. 35-39). Muscles act as protein reservoirs in the acute setting, delivering amino acids to the splanchnic area through activation of muscle protein catabolism. Insufficient protein intake to satisfy daily requirements leads to negative protein balance and results in skeletal muscle atrophy, impaired muscle growth, and functional decline (Deer, R. R. and Volpi, E., 2018. Nutrients, 10 (3), p. 378). Observational studies indicate that decreased protein intake appears to be associated with worse clinical outcomes in critically ill patients (Alberda C, et al. Intensive Care Med 2009; 35 (10): 1728-1737; and Weijs P J, et al. Crit Care 2014; 18 (6): 701).

Preclinical studies in sepsis have shown that specific amino acid supplementation decreased muscle protein breakdown and increased muscle protein synthesis, thereby improving recovery. However, previous trials assessing a balanced set of amino acids have failed to improve outcomes in the critically ill (Doig G S, et al. Intensive Care Med 2015; 41 (7): 1197-1208; and Ferrie S, et al. JPEN J Parenter Enteral Nutr 2016; 40 (6): 795-805).

Moreover, amino acid supplementation can actually worsen outcomes in the critically ill. Supplementation by glutamine in the critically ill was not associated with an improved outcome and was even associated with an increased risk of death in the sickest patients (Heyland D, et al N Engl J Med 2013; 368 (16): 1489-1497). Supplementation by arginine may also be associated with an increased risk of death in the sickest patients (Marik P E, et al. Intensive Care Med 2008; 34 (11): 1980-1990).

### SUMMARY OF THE INVENTION

The inventors have surprisingly shown that a unique combination of amino acids can protect body pools of glutamine and arginine without enriching the enteral feed directly with these two amino acids.

The supply of five specific amino acids (leucine, cysteine, threonine, serine and proline), given as a supplement on top of usual enteral nutrition of ICU patients was able to cover at the same time amino acid requirements of the splanchnic area and the muscle, which may promote arginine and glutamine stabilization. Glutamine, a major fuel for the enterocytes may be spared since the gut barrier and function is quickly restored. Proline may be an alternative precursor for arginine synthesis, sparing glutamine and vice versa, since arginine and glutamine synthesis are interlinked.

The combination of leucine, cysteine, threonine, serine and proline may allow the body to sustain its metabolic needs for arginine and glutamine, without relying on muscle or exogenous direct supply for these two amino acids. The inventors have shown that the amino acid combination can decrease muscle protein breakdown and was associated with improved skeletal muscle and diaphragm functions.

These benefits contribute to favorable clinical outcome compared to an isocaloric enteral feed, including a shorter time of ventilation and shorter stay in ICU, as well normalisation of cholesterol level, which is a positive prognostic in ICU patients.

In one aspect, the present invention provides an amino acid composition comprising threonine, serine, proline, leucine, and cysteine.

In another aspect, the present invention provides an amino acid composition comprising threonine, serine, proline, leucine, and cysteine for use in preventing and/or reducing glutamine deficiency and/or arginine deficiency in a subject.

In another aspect, the present invention provides an amino acid composition comprising threonine, serine, proline, leucine, and cysteine for use in preventing and/or reducing muscle loss in a subject. The subject may be at risk of or has glutamine deficiency and/or arginine deficiency.

In some embodiments, the subject is a critically ill patient. In some embodiments, the subject has a condition selected from one or more of: sepsis, acute respiratory failure, trauma, surgery, shock, pancreatitis, severe burns, mucositis, inflammatory bowel disease, distorted gut barrier, intestinal mucosal lesions, and short-bowel syndrome. In some embodiments, the subject is a pre-term infant.

The amino acid composition may improve muscle mass and/or strength. The amino acid composition may improve skeletal muscle mass and/or strength, and/or diaphragm muscle strength. The amino acid composition may promote gut healing. The amino acid composition may promote recovery of functional enterocyte mass and/or promote recovery of gut barrier structure and/or function.

The amino acid composition may be provided in any suitable form. The amino acid composition may be in the form of a nutritional composition, supplement, or fortifier. In some embodiments, the amino acid composition is in the form of a nutritional composition. In some embodiments, the amino acid composition is in the form of an enteral nutritional composition.

The amino acid composition may be administered by any suitable method (e.g. by enteral, oral, or parenteral administration). Suitably, the amino acid composition may be administered by enteral administration. In some embodiments, the amino acid composition is administered by a feeding tube.

The subject may be administered each of threonine, serine, proline, leucine, and cysteine in an amount of about 0.02 g/kg/day or more, about 0.03 g/kg/day or more, or about 0.04 g/kg/day or more. The subject may be administered each of threonine, serine, proline, leucine, and cysteine in an amount of about 0.5 g/kg/day or less, about 0.4 g/kg/day or less, or about 0.3 g/kg/day or less. The subject may be administered each of threonine, serine, proline, leucine, and cysteine in an amount of from about 0.02 g/kg/day to about 0.5 g/kg/day, from about 0.03 g/kg/day to about 0.4 g/kg/day, or from about 0.04 g/kg/day to about 0.3 g/kg/day. In some embodiments, the subject is administered: (i) from about 0.06 g/kg/day to about 0.5 g/kg/day, from about 0.08 g/kg/day to about 0.4 g/kg/day, or from about 0.1 g/kg/day to about 0.3 g/kg/day threonine; (ii) from about 0.04 g/kg/day to about 0.4 g/kg/day, from about 0.06 g/kg/day to about 0.3 g/kg/day, or from about 0.08 g/kg/day to about 0.2 g/kg/day serine; (iii) from about 0.02 g/kg/day to about 0.4 g/kg/day, from about 0.03 g/kg/day to about 0.3 g/kg/day, or from about 0.04 g/kg/day to about 0.2 g/kg/day proline; (iv) from about 0.04 g/kg/day to about 0.5 g/kg/day, from about 0.06 g/kg/day to about 0.4 g/kg/day, or from about 0.08 g/kg/day to about 0.3 g/kg/day leucine; and/or (v) from about 0.02 g/kg/day to about 0.4 g/kg/day, from about 0.04 g/kg/day to about 0.3 g/kg/day, or from about 0.06 g/kg/day to about 0.2 g/kg/day cysteine.

The subject may be administered each of threonine, serine, proline, leucine, and cysteine in an amount of about 1% or more, about 2% or more, or about 3% or more as a percentage of total protein. The subject may be administered each of threonine, serine, proline, leucine, and cysteine in an amount of about 18% or less, about 16% or less, or about 14% or less as a percentage of total protein. The subject may be administered each of threonine, serine, proline, leucine, and cysteine in an amount of from about 1% to about 18%, from about 2% to about 16%, or from about 3% to about 14% as a percentage of total protein. In some embodiments, the subject is administered: (i) from about 5% to about 16%, from about 6% to about 14%, or from about 7% to about 12% threonine as a percentage of total protein; (ii) from about 4% to about 16%, from about 5% to about 14%, or from about 6% to about 12% serine as a percentage of total protein; (iii) from about 1% to about 16%, from about 2% to about 14%, or from about 3% to about 12% proline as a percentage of total protein; (iv) from about 3% to about 18%, from about 4% to about 16%, or from about 5% to about 14% leucine as a percentage of total protein; and/or (v) from about 2% to about 10%, from about 3% to about 8%, or from about 4% to about 6% cysteine as a percentage of total protein.

The amino acid composition may comprise: (i) a weight ratio of threonine:serine of from about 0.5:1 to about 1.5:1; (ii) a weight ratio of threonine:proline of from about 0.5:1 to about 2.5:1; (iii) a weight ratio of threonine:leucine of from about 0.5:1 to about 1.5:1; (iv) a weight ratio of threonine:cysteine of from about 1:1 to about 2.5:1; (v) a weight ratio of serine:proline of from about 0.5:1 to about 2:1; (vi) a weight ratio of serine:leucine of from about 0.5:1 to about 2:1; (vii) a weight ratio of serine:cysteine of from about 1:1 to about 2.5:1; (viii) a weight ratio of proline:leucine of from about 0.5:1 to about 1.5:1; (ix) a weight ratio of proline:cysteine of from about 0.5:1 to about 2.5:1; and/or (x) a weight ratio of leucine:cysteine of from about 1:1 to about 3:1.

The amino acid composition may comprise each of threonine, serine, proline, leucine, and cysteine in an amount of about 0.5 g/1000 kcal or more, about 1 g/1000 kcal or more, or about 1.5 g/1000 kcal or more. The amino acid composition may comprise each of threonine, serine, proline, leucine, and cysteine in an amount of about 20 g/1000 kcal or less, about 15 g/1000 kcal or less, or about 12 g/1000 kcal or less. The amino acid composition may comprise each of threonine, serine, proline, leucine, and cysteine in an amount of from about 0.5 g/1000 kcal to about 20 g/1000 kcal, from about 1 g/1000 kcal to about 15 g/1000 kcal, or from about 1.5 g/1000 kcal to about 12 g/1000 kcal. In some embodiments, the amino acid composition comprises: (i) from about 1 g/1000 kcal to about 20 g/1000 kcal, from about 2 g/1000 kcal to about 15 g/1000 kcal, or from about 3 g/1000 kcal to about 10 g/1000 kcal threonine; (ii) from about 1 g/1000 kcal to about 16 g/1000 kcal, from about 2 g/1000 kcal to about 12 g/1000 kcal, or from about 3 g/1000 kcal to about 8 g/1000 kcal serine; (iii) from about 0.5 g/1000 kcal to about 16 g/1000 kcal, from about 1 g/1000 kcal to about 12 g/1000 kcal, or from about 1.5 g/1000 kcal to about 8 g/1000 kcal proline; (iv) from about 1 g/1000 kcal to about 20 g/1000 kcal, from about 1.5 g/1000 kcal to about 16 g/1000 kcal, or from about 2 g/1000 kcal to about 12 g/1000 kcal leucine; and/or (v) from about 1 g/1000 kcal to about 10 g/1000 kcal, from about 1.5 g/1000 kcal to about 7.5 g/1000 kcal, or from about 2 g/1000 kcal to about 5 g/1000 kcal cysteine.

The amino acid composition may comprise each of threonine, serine, proline, leucine, and cysteine in an amount of about 1% or more, about 2% or more, or about 3% or more as a percentage of total protein. The amino acid composition may comprise each of threonine, serine, proline, leucine, and cysteine in an amount of about 18% or less, about 16% or less, or about 14% or less as a percentage of total protein. The amino acid composition may comprise each of threonine, serine, proline, leucine, and cysteine in an amount of from about 1% to about 18%, from about 2% to about 16%, or from about 3% to about 14% as a percentage of total protein. In some embodiments, the amino acid composition comprises: (i) from about 5% to about 16%, from about 6% to about 14%, or from about 7% to about 12% threonine as a percentage of total protein; (ii) from about 4% to about 16%, from about 5% to about 14%, or from about 6% to about 12% serine as a percentage of total protein; (iii) from about 1% to about 16%, from about 2% to about 14%, or from about 3% to about 12% proline as a percentage of total protein; (iv) from about 3% to about 18%, from about 4% to about 16%, or from about 5% to about 14% leucine as a percentage of total protein; and/or (v) from about 2% to about 10%, from about 3% to about 8%, or from about 4% to about 6% cysteine as a percentage of total protein.

The amino acid composition may comprise any other suitable components. Suitably, the amino acid composition may further comprise one or more additional amino acids. In some embodiments, the amino acid composition further comprises aspartate, isoleucine, or valine, or any combination thereof. Suitably, the amino acid composition may comprise from about 1000 kcal/L to about 2000 kcal/L. Suitably, the amino acid composition may comprise protein. In some embodiments, the amino acid composition comprises from about 50 g/L to about 120 g/L protein. Suitably, the amino acid composition may comprise lipid (fat). In some embodiments, the amino acid composition comprises from about 20 g/L to about 100 g/L lipid. Suitably, the amino acid composition may comprise carbohydrate. In some embodiments, the amino acid composition comprises from about 100 g/L to about 200 g/L carbohydrate.

In some embodiments, the amino acid composition does not comprise added glutamine and/or arginine.

In another aspect, the present invention provides a method of producing a nutritional composition, comprising: (i) providing a base nutritional composition; and (ii) adding threonine, serine, proline, leucine, and cysteine to the base nutritional composition.

The nutritional composition manufactured by the method of the present invention may be any nutritional composition according to the present invention. In some embodiments, the nutritional composition is an enteral nutritional composition.

In another aspect, the present invention provides a combination of threonine, serine, proline, leucine, and cysteine for use in preventing and/or reducing glutamine deficiency and/or arginine deficiency in a subject.

In another aspect, the present invention provides a combination of threonine, serine, proline, leucine, and cysteine for use in preventing and/or reducing muscle loss in a subject. The subject may be at risk of or may have a glutamine deficiency and/or an arginine deficiency.

The combination of threonine, serine, proline, leucine, and cysteine may be administered by any method described herein. The threonine, serine, proline, leucine, and cysteine may be administered in the form of an amino acid composition according to the present invention.

### DESCRIPTION OF DRAWINGS

FIG. 1—Study design

Baseline visit (V1) was performed within 72 hours of admission. Follow up visits (V2 to V7) occurred 7, 14, 21, 60, 180 and 365 days after randomisation.

FIG. 2—Plasma citrulline (nmol/mL)

Mean changes from baseline at days 7, 14, 21 and 60, using a restricted maximum likelihood (REML)-based repeated measures approach.

FIG. 3—Muscle mass and strength

(A) Indexed muscle volume of the anterior compartment of the quadriceps (cc/kg); and (B) Diaphragm strength (cm H₂O). Mean changes from baseline at days 7, 14, 21 and 60, using a restricted maximum likelihood (REML)-based repeated measures approach.

FIG. 4—Arginine and glutamine (μmol/L)

Plasma concentration of: (A) Arginine; and (B) Glutamine. Mean changes from baseline at days 7, 14, 21 and 60, using a restricted maximum likelihood (REML)-based repeated measures approach.

FIG. 5—Total cholesterol (mmol/L)

Mean changes from baseline at days 7, 14, 21 and 60, using a restricted maximum likelihood (REML)-based repeated measures approach.

FIG. 6—ICU length of stay

The median number of ICU free days until day 21 was 9.5 [0; 13] days in the interventional arm vs 6.0 [0; 12] in the placebo arm (P=0.457)

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

All publications mentioned in the specification are herein incorporated by reference.

### Amino Acid Composition

In one aspect, the present invention provides an amino acid composition comprising threonine, serine, proline, leucine, and cysteine.

An “amino acid composition” may refer to a combination, mix, or blend, of amino acids provided in a form suitable for administration (e.g. enterally, orally, or parenterally) to a subject. The term “amino acid composition” may therefore be used interchangeably with any of the terms “amino acid combination”, “amino acid mix”, or “amino acid blend”.

### Amino Acids

The amino acid composition may comprise the amino acids in any form suitable for administration (e.g. enterally, orally, or parenterally) to a subject. For example, the amino acids may be provided in the form of free amino acids or a salt thereof, oligopeptides, polypeptides, proteins, amino acid precursors or any combination thereof.

As used herein, “free amino acids” may refer to amino acid monomers, which are not part of an oligopeptide, polypeptide, or protein. Amino acid salts may include any physiologically acceptable salt, such as hydrochloride, sodium, potassium, calcium, and magnesium salts.

As used herein, “oligopeptides” may refer to short chains of amino acid monomers (e.g. 2 to 20 amino acid monomers) linked via peptide bonds and can include dipeptides, tripeptides, tetrapeptides, and pentapeptides. The oligopeptides may be enriched for one or more amino acids or consist solely of a single type of amino acid.

As used herein, “polypeptides” and “proteins” may refer to longer chains of amino acids (e.g. greater than about 20 amino acids and 50 amino acids, respectively). The amino acids may be, partially, or entirely, in the form of proteins. The polypeptides or proteins may be enriched for one or more amino acids.

As used herein, “amino acid precursors” may refer to compounds that give the amino acid after some reaction(s) (usually hydrolysis).

In some embodiments, the amino acid composition comprises one or more of threonine, serine, proline, leucine, and cysteine as a free amino acid or a salt thereof and/or in the form of oligopeptides, polypeptides, or protein. In some embodiments, the amino acid composition comprises each of threonine, serine, proline, leucine, and cysteine as a free amino acid or a salt thereof and/or in the form of oligopeptides, polypeptides, or protein.

In some embodiments, the amino acid composition comprises one or more of threonine, serine, proline, leucine, and cysteine partially, or wholly, in the form of proteins. In some embodiments, the amino acid composition comprises each of threonine, serine, proline, leucine, and cysteine partially, or wholly, in the form of proteins.

### Threonine

Threonine (also known as L-threonine, symbol Thr or T) is an amino acid having the following formula:

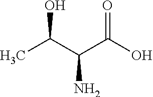

The amino acid composition may comprise threonine in any suitable form, for example in the form of a free amino acid, salt (e.g. hydrochloride, sodium, potassium, calcium, and magnesium salts), oligopeptide, polypeptide, protein, or amino acid precursor.

In some embodiments, the amino acid composition comprises threonine as a free amino acid or salt thereof. In some embodiments, the amino acid composition comprises threonine in the form of an oligopeptide, polypeptide, or protein. In some embodiments, the amino acid composition comprises threonine partially, or wholly, in the form of proteins.

### Serine

Serine (also known as L-serine, symbol Ser or S) is an amino acid having the following formula:

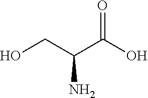

The amino acid composition may comprise serine in any suitable form, for example in the form of a free amino acid, salt (e.g. hydrochloride, sodium, potassium, calcium, and magnesium salts), oligopeptide, polypeptide, protein, or amino acid precursor.

In some embodiments, the amino acid composition comprises serine as a free amino acid or salt thereof. In some embodiments, the amino acid composition comprises serine in the form of an oligopeptide, polypeptide, or protein. In some embodiments, the amino acid composition comprises serine partially, or wholly, in the form of proteins.

### Proline

Proline (symbol Pro or P) is an amino acid having the following formula:

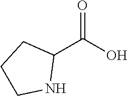

The amino acid composition may comprise proline in any suitable form, for example in the form of a free amino acid, salt (e.g. hydrochloride, sodium, potassium, calcium, and magnesium salts), oligopeptide, polypeptide, protein, or amino acid precursor.

In some embodiments, the amino acid composition comprises proline as a free amino acid or salt thereof. In some embodiments, the amino acid composition comprises proline in the form of an oligopeptide, polypeptide, or protein. In some embodiments, the amino acid composition comprises proline partially, or wholly, in the form of proteins.

### Leucine

Leucine (also known as L-leucine, symbol Leu or L) is an amino acid having the following formula:

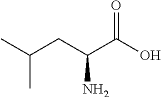

The amino acid composition may comprise leucine in any suitable form, for example in the form of a free amino acid, salt (e.g. hydrochloride, sodium, potassium, calcium, and magnesium salts), oligopeptide, polypeptide, protein, or amino acid precursor.

In some embodiments, the amino acid composition comprises leucine as a free amino acid or salt thereof. In some embodiments, the amino acid composition comprises leucine in the form of an oligopeptide, polypeptide, or protein. In some embodiments, the amino acid composition comprises leucine partially, or wholly, in the form of proteins.

### Cysteine

Cysteine (also known as L-cysteine, symbol Cys or C) is an amino acid having the following formula:

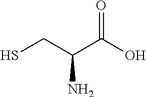

The amino acid composition may comprise cysteine in any suitable form, for example in the form of a free amino acid, salt (e.g. hydrochloride, sodium, potassium, calcium, and magnesium salts), oligopeptide, polypeptide, or protein.

In some embodiments, the amino acid composition comprises cysteine as a free amino acid or salt thereof. In some embodiments, the amino acid composition comprises cysteine in the form of an oligopeptide, polypeptide, or protein. In some embodiments, the amino acid composition comprises cysteine partially, or wholly, in the form of proteins. In some embodiments, the cysteine may be provided in the form of a cysteine precursor. Suitable cysteine precursors are, for example, cystathionine and N-acetylcysteine.

### Other Amino Acids

The amino acid composition may further comprise any other suitable amino acid. For example, the amino acid composition may further comprise aspartate, isoleucine, valine, arginine, or glutamine, or any combination thereof.

The amino acid composition may comprise aspartate, isoleucine, valine, arginine, and/or glutamine in any suitable form, for example in the form of a free amino acid, salt (e.g. hydrochloride, sodium, potassium, calcium, and magnesium salts), oligopeptide, polypeptide, protein, or amino acid precursor.

In some embodiments, the amino acid composition comprises aspartate, isoleucine, valine, arginine, and/or glutamine as a free amino acid or salt thereof. In some embodiments, the amino acid composition comprises aspartate, isoleucine, valine, arginine, and/or glutamine in the form of an oligopeptide, polypeptide, or protein. In some embodiments, the amino acid composition comprises aspartate, isoleucine, valine, arginine, and/or glutamine partially, or wholly, in the form of proteins.

The amino acid composition may comprise glutamine and/or arginine, although the amino acid composition of the present invention allows the body to sustain its metabolic needs for arginine and glutamine without relying on exogenous direct supply for these two amino acids. Suitably, the amino acid composition may comprise:
(i) a weight ratio of glutamine:threonine of about 2:1 or less, about 1:1 or less, about 0.5:1 or less, about 0.4:1 or less, about 0.3:1 or less, about 0.2:1 or less, or about 0.1:1 or less;
(ii) a weight ratio of glutamine:serine of about 2:1 or less, about 1:1 or less, about 0.5:1 or less, about 0.4:1 or less, about 0.3:1 or less, about 0.2:1 or less, or about 0.1:1 or less;
(iii) a weight ratio of glutamine:proline of about 2:1 or less, about 1:1 or less, about 0.5:1 or less, about 0.4:1 or less, about 0.3:1 or less, about 0.2:1 or less, or about 0.1:1 or less;
(iv) a weight ratio of glutamine:leucine of about 2:1 or less, about 1:1 or less, about 0.5:1 or less, about 0.4:1 or less, about 0.3:1 or less, about 0.2:1 or less, or about 0.1:1 or less;
(v) a weight ratio of glutamine:cysteine of about 2:1 or less, about 1:1 or less, about 0.5:1 or less, about 0.4:1 or less, about 0.3:1 or less, about 0.2:1 or less, or about 0.1:1 or less;
(vi) a weight ratio of arginine:threonine of about 1:1 or less, about 0.5:1 or less, about 0.4:1 or less, about 0.3:1 or less, about 0.2:1 or less, or about 0.1:1 or less;
(vii) a weight ratio of arginine:serine of about 1:1 or less, about 0.5:1 or less, about 0.4:1 or less, about 0.3:1 or less, about 0.2:1 or less, or about 0.1:1 or less;
(viii) a weight ratio of arginine:proline of about 1:1 or less, about 0.5:1 or less, about 0.4:1 or less, about 0.3:1 or less, about 0.2:1 or less, or about 0.1:1 or less;
(ix) a weight ratio of arginine:leucine of about 1:1 or less, about 0.5:1 or less, about 0.4:1 or less, about 0.3:1 or less, about 0.2:1 or less, or about 0.1:1 or less; and/or
(x) a weight ratio of arginine:cysteine of about 1:1 or less, about 0.5:1 or less, about 0.4:1 or less, about 0.3:1 or less, about 0.2:1 or less, or about 0.1:1 or less.

In some embodiments, the amino acid composition does not comprise added glutamine and/or arginine. The term “added glutamine and/or arginine” does not include glutamine and/or arginine present in minor amounts as natural constituents of other ingredients of the amino acid composition.

In some embodiments, the amino acid composition does not comprise glutamine and/or arginine as a free amino acid, or as a salt thereof. In some embodiments, the amino acid composition is substantially devoid of glutamine and/or arginine. In some embodiments, the amino acid composition does not comprise glutamine and/or arginine.

### Weight Ratios

The amino acids may be present in any suitable ratio.

Suitably, threonine, serine, proline, leucine, and cysteine may be present in a weight ratio (threonine:serine:proline:leucine:cysteine) of about 0.1-10:0.1-10:0.1-10:0.1-10:0.1-10, or about 0.2-8:0.2-8:0.2-8:0.2-8:0.2-8, or about 0.3-6:0.3-6:0.3-6:0.3-6:0.3-6, or about 0.4-5:0.4-5:0.4-5:0.4-5:0.4-5, or about 0.5-3:0.5-3:0.5-3:0.5-3:0.5-3.

In some embodiments, the amino acid composition comprises:
(i) a weight ratio of threonine:serine of from about 0.1:1 to about 10:1, from about 0.2:1 to about 5:1, or from about 0.5:1 to about 1.5:1;
(ii) a weight ratio of threonine:proline of from about 0.1:1 to about 10:1, from about 0.2:1 to about 5:1, or of from about 0.5:1 to about 2.5:1;
(iii) a weight ratio of threonine:leucine of from about 0.1:1 to about 10:1, from about 0.2:1 to about 5:1, or of from about 0.5:1 to about 1.5:1;
(iv) a weight ratio of threonine:cysteine of from about 0.1:1 to about 10:1, from about 0.2:1 to about 5:1, or of from about 1:1 to about 2.5:1;
(v) a weight ratio of serine:proline of from about 0.1:1 to about 10:1, from about 0.2:1 to about 5:1, or of from about 0.5:1 to about 2:1;
(vi) a weight ratio of serine:leucine of from about 0.1:1 to about 10:1, from about 0.2:1 to about 5:1, or of from about 0.5:1 to about 2:1;
(vii) a weight ratio of serine:cysteine of from about 0.1:1 to about 10:1, from about 0.2:1 to about 5:1, or of from about 1:1 to about 2.5:1;
(viii) a weight ratio of proline:leucine of from about 0.1:1 to about 10:1, from about 0.2:1 to about 5:1, or of from about 0.5:1 to about 1.5:1;
(ix) a weight ratio of proline:cysteine of from about 0.1:1 to about 10:1, from about 0.2:1 to about 5:1, or of from about 0.5:1 to about 2.5:1; and/or
(x) a weight ratio of leucine:cysteine of from about 0.1:1 to about 10:1, from about 0.2:1 to about 5:1, or of from about 1:1 to about 3:1.

### Form of Composition

The amino acid composition may be provided in any suitable form for administration to a subject. For example, the amino acid composition can be in a liquid, solid (e.g. powder), or gelatinous form.

The amino acid composition may be provided in a form suitable for enteral, oral, or parenteral administration. In some embodiments, the amino acid composition is provided in a form suitable for enteral administration.

In some embodiments, the amino acid composition is provided in a liquid form. Providing the amino acid composition in the form of a liquid may be more suitable for use as a nutritional composition.

In some embodiments, the amino acid composition is provided in a solid (e.g. powder) form. Providing the amino acid composition in the form of a solid (e.g. a powder) may be more suitable for use as a supplement or fortifier.

The amino acid composition may be provided in the form of a nutritional composition, supplement or fortifier. In some embodiments, the amino acid composition is provided in the form of an enteral nutritional composition. Nutritional compositions, supplements, and fortifiers are described in more detail in the sections entitled “Nutritional composition” and “Supplement or fortifier”.

### Nutritional Composition

In one aspect, the present invention provides a nutritional composition comprising threonine, serine, proline, leucine, and cysteine.

A “nutritional composition” may refer to a composition which nourishes a subject. The nutritional composition may be prepared in any suitable manner. The nutritional composition is not particularly limited as long as it is suitable for administration (e.g. enteral, oral, or parenteral administration). Examples of suitable nutritional compositions include tube feeds, foodstuffs, drinks, and drug bases.

The nutritional composition according to the invention may be an enteral nutritional composition.

An “enteral nutritional composition” may refer to a nutritional composition that involves the gastrointestinal tract for its administration. For example, an enteral nutritional composition may suitable for administration by tube feeding. An enteral nutritional composition is preferably for patients who have a condition which prevents eating a regular diet by mouth, but in which their gastrointestinal tract is still able to function.

In some embodiments, the nutritional composition is a tube feed. A “tube feed” may refer to a liquid enteral nutritional composition that is suitable for administration by tube feeding.

### Amount of Amino Acids

The nutritional composition may comprise any suitable amount of threonine, serine, proline, leucine, and cysteine. For example, suitable weight ratios are described in the section entitled “Amino acid composition” and suitable doses are described in the section entitled “Use as a medicament”.

### Mass Per 1000 Kcal

The nutritional composition may comprise each of threonine, serine, proline, leucine, and cysteine in an amount of about 0.5 g/1000 kcal or more, about 0.6 g/1000 kcal or more, about 0.7 g/1000 kcal or more, about 0.8 g/1000 kcal or more, about 0.9 g/1000 kcal or more, about 1.0 g/1000 kcal or more, about 1.1 g/1000 kcal or more, about 1.2 g/1000 kcal or more, about 1.3 g/1000 kcal or more, about 1.4 g/1000 kcal or more, about 1.5 g/1000 kcal or more, about 2 g/1000 kcal or more, about 3 g/1000 kcal or more, about 4 g/1000 kcal or more, about 5 g/1000 kcal or more, about 6 g/1000 kcal or more, or about 7 g/1000 kcal or more.

The nutritional composition may comprise each of threonine, serine, proline, leucine, and cysteine in an amount of about 20 g/1000 kcal or less, about 19 g/1000 kcal or less, about 18 g/1000 kcal or less, about 17 g/1000 kcal or less, about 16 g/1000 kcal or less, about 15 g/1000 kcal or less, about 14 g/1000 kcal or less, about 13 g/1000 kcal or less, or about 12 g/1000 kcal or less, about 11 g/1000 kcal or less, about 10 g/1000 kcal or less.

The nutritional composition may comprise each of threonine, serine, proline, leucine, and cysteine in an amount of from about 0.5 g/1000 kcal to about 20 g/1000 kcal, 0.6 g/1000 kcal to about 20 g/1000 kcal, 0.7 g/1000 kcal to about 20 g/1000 kcal, 0.8 g/1000 kcal to about 20 g/1000 kcal, 0.9 g/1000 kcal to about 19 g/1000 kcal, 1.0 g/1000 kcal to about 18 g/1000 kcal, 1.1 g/1000 kcal to about 17 g/1000 kcal, 1.2 g/1000 kcal to about 16 g/1000 kcal, from about 1.3 g/1000 kcal to about 15 g/1000 kcal, from about 1.4 g/1000 kcal to about 14 g/1000 kcal, from about 1.5 g/1000 kcal to about 13 g/1000 kcal, from about 2 g/1000 kcal to about 12 g/1000 kcal, from about 2 g/1000 kcal to about 11 g/1000 kcal, or from about 2 g/1000 kcal to about 10 g/1000 kcal.

Suitably, the nutritional composition may comprise threonine in an amount of about 1 g/1000 kcal or more, about 2 g/1000 kcal or more, or about 3 g/1000 kcal or more.

Suitably, the nutritional composition may comprise threonine in an amount of about 20 g/1000 kcal or less, about 15 g/1000 kcal or less, or about 10 g/1000 kcal or less.

Suitably, the nutritional composition may comprise threonine in an amount of from about 1 g/1000 kcal to about 20 g/1000 kcal, from about 2 g/1000 kcal to about 15 g/1000 kcal, or from about 3 g/1000 kcal to about 10 g/1000 kcal.

Suitably, the nutritional composition may comprise serine in an amount of about 1 g/1000 kcal or more, about 2 g/1000 kcal or more, or about 3 g/1000 kcal or more.

Suitably, the nutritional composition may comprise serine in an amount of about 16 g/1000 kcal or less, about 12 g/1000 kcal or less, or about 8 g/1000 kcal or less.

Suitably, the nutritional composition may comprise serine in an amount of from about 1 g/1000 kcal to about 16 g/1000 kcal, from about 2 g/1000 kcal to about 12 g/1000 kcal, or from about 3 g/1000 kcal to about 8 g/1000 kcal.

Suitably, the nutritional composition may comprise proline in an amount of about 0.5 g/1000 kcal or more, about 1 g/1000 kcal or more, or about 1.5 g/1000 kcal or more.

Suitably, the nutritional composition may comprise proline in an amount of about 16 g/1000 kcal or less, about 12 g/1000 kcal or less, or about 8 g/1000 kcal or less.

Suitably, the nutritional composition may comprise proline in an amount of from about 0.5 g/1000 kcal to about 16 g/1000 kcal, from about 1 g/1000 kcal to about 12 g/1000 kcal, or from about 1.5 g/1000 kcal to about 8 g/1000 kcal.

Suitably, the nutritional composition may comprise leucine in an amount of about 1 g/1000 kcal or more, about 1.5 g/1000 kcal or more, or about 2 g/1000 kcal or more.

Suitably, the nutritional composition may comprise leucine in an amount of about 20 g/1000 kcal or less, about 16 g/1000 kcal or less, or about 12 g/1000 kcal or less.

Suitably, the nutritional composition may comprise leucine in an amount of from about 1 g/1000 kcal to about 20 g/1000 kcal, from about 1.5 g/1000 kcal to about 16 g/1000 kcal, or from about 2 g/1000 kcal to about 12 g/1000 kcal.

Suitably, the nutritional composition may comprise cysteine in an amount of about 1 g/1000 kcal or more, about 1.5 g/1000 kcal or more, or about 2 g/1000 kcal or more.

Suitably, the nutritional composition may comprise cysteine in an amount of about 10 g/1000 kcal or less, about 7.5 g/1000 kcal or less, or about 5 g/1000 kcal or less.

Suitably, the nutritional composition may comprise cysteine in an amount of from about 1 g/1000 kcal to about 10 g/1000 kcal, from about 1.5 g/1000 kcal to about 7.5 g/1000 kcal, or from about 2 g/1000 kcal to about 5 g/1000 kcal.

In some embodiments, the nutritional composition comprises:
(i) threonine in an amount of from about 1 g/1000 kcal to about 20 g/1000 kcal, from about 2 g/1000 kcal to about 15 g/1000 kcal, or from about 3 g/1000 kcal to about 10 g/1000 kcal;
(ii) serine in an amount of from about 1 g/1000 kcal to about 16 g/1000 kcal, from about 2 g/1000 kcal to about 12 g/1000 kcal, or from about 3 g/1000 kcal to about 8 g/1000 kcal;
(iii) proline in an amount of from about 0.5 g/1000 kcal to about 16 g/1000 kcal, from about 1 g/1000 kcal to about 12 g/1000 kcal, or from about 1.5 g/1000 kcal to about 8 g/1000 kcal;
(iv) leucine in an amount of from about 1 g/1000 kcal to about 20 g/1000 kcal, from about 1.5 g/1000 kcal to about 16 g/1000 kcal, or from about 2 g/1000 kcal to about 12 g/1000 kcal; and
(v) cysteine in an amount of from about 1 g/1000 kcal to about 10 g/1000 kcal, from about 1.5 g/1000 kcal to about 7.5 g/1000 kcal, or from about 2 g/1000 kcal to about 5 g/1000 kcal.

### Percentage of Total Protein

The nutritional composition may comprise each of threonine, serine, proline, leucine, and cysteine in an amount of about 1% or more, about 2% or more, about 3% or more, about 4% or more, or about 5% or more as a percentage of total protein.

The nutritional composition may comprise each of threonine, serine, proline, leucine, and cysteine in an amount of about 18% or less, about 16% or less, about 14% or less, about 12% or less, or about 10% or less as a percentage of total protein.

The nutritional composition may comprise each of threonine, serine, proline, leucine, and cysteine in an amount of from about 1% to about 18%, from about 2% to about 16%, from about 3% to about 14%, from about 4% to about 12%, of from about 5% to about 12%.

Suitably, the nutritional composition may comprise threonine in an amount of about 5% or more, about 6% or more, or about 7% or more as a percentage of total protein.

Suitably, the nutritional composition may comprise threonine in an amount of about 16% or less, about 14% or less, or about 12% or less as a percentage of total protein.

Suitably, the nutritional composition may comprise threonine in an amount of from about 5% to about 16%, from about 6% to about 14%, or from about 7% to about 12% as a percentage of total protein.

Suitably, the nutritional composition may comprise serine in an amount of about 4% or more, about 5% or more, or about 6% or more as a percentage of total protein.

Suitably, the nutritional composition may comprise serine in an amount of about 16% or less, about 14% or less, or about 12% or less as a percentage of total protein.

Suitably, the nutritional composition may comprise serine in an amount of from about 4% to about 16%, from about 5% to about 14%, or from about 6% to about 12% as a percentage of total protein.

Suitably, the nutritional composition may comprise proline in an amount of about 1% or more, about 2% or more, or about 3% or more as a percentage of total protein.

Suitably, the nutritional composition may comprise proline in an amount of about 16% or less, about 14% or less, or about 12% or less as a percentage of total protein.

Suitably, the nutritional composition may comprise proline in an amount of from about 1% to about 16%, from about 2% to about 14%, or from about 3% to about 12% as a percentage of total protein.

Suitably, the nutritional composition may comprise leucine in an amount of about 3% or more, about 4% or more, or about 5% or more as a percentage of total protein.

Suitably, the nutritional composition may comprise leucine in an amount of about 18% or less, about 16% or less, or about 14% or less as a percentage of total protein.

Suitably, the nutritional composition may comprise leucine in an amount of from about 3% to about 18%, from about 4% to about 16%, or from about 5% to about 14% as a percentage of total protein.

Suitably, the nutritional composition may comprise cysteine in an amount of about 2% or more, about 3% or more, or about 4% or more as a percentage of total protein.

Suitably, the nutritional composition may comprise cysteine in an amount of about 10% or less, about 8% or less, or about 6% or less as a percentage of total protein.

Suitably, the nutritional composition may comprise cysteine in an amount of about 2% to about 10%, from about 3% to about 8%, or from about 4% to about 6% as a percentage of total protein.

In some embodiments, the nutritional composition comprises:
(i) threonine in an amount of from about 5% to about 16%, from about 6% to about 14%, or from about 7% to about 12% as a percentage of total protein;
(ii) serine in an amount of from about 4% to about 16%, from about 5% to about 14%, or from about 6% to about 12% as a percentage of total protein;
(iii) proline in an amount of from about 1% to about 16%, from about 2% to about 14%, or from about 3% to about 12% as a percentage of total protein;
(iv) leucine in an amount of from about 3% to about 18%, from about 4% to about 16%, or from about 5% to about 14% as a percentage of total protein; and
(v) cysteine in an amount of from about 2% to about 10%, from about 3% to about 8%, or from about 4% to about 6% as a percentage of total protein.

### Other Components

The nutritional composition may comprise any other suitable components. For example, suitable amino acids are described in the section entitled “Amino acid composition”.

### Other Amino Acids

The amino acid composition may further comprise one or more additional amino acids. In some embodiments, the amino acid composition further comprises aspartate, isoleucine, or valine, or any combination thereof.

Suitably, the nutritional composition may comprise isoleucine in an amount of about 1 g/1000 kcal or more, about 1.5 g/1000 kcal or more, or about 2 g/1000 kcal or more.

Suitably, the nutritional composition may comprise isoleucine in an amount of about 10 g/1000 kcal or less, about 8 g/1000 kcal or less, or about 5 g/1000 kcal or less.

Suitably, the nutritional composition may comprise isoleucine in an amount of from about 1 g/1000 kcal to about 10 g/1000 kcal, from about 1.5 g/1000 kcal to about 8 g/1000 kcal, or from about 2 g/1000 kcal to about 5 g/1000 kcal.

Suitably, the nutritional composition may comprise valine in an amount of about 1 g/1000 kcal or more, about 1.5 g/1000 kcal or more, or about 2 g/1000 kcal or more.

Suitably, the nutritional composition may comprise valine in an amount of about 10 g/1000 kcal or less, about 8 g/1000 kcal or less, or about 5 g/1000 kcal or less.

Suitably, the nutritional composition may comprise valine in an amount of from about 1 g/1000 kcal to about 10 g/1000 kcal, from about 1.5 g/1000 kcal to about 8 g/1000 kcal, or from about 2 g/1000 kcal to about 5 g/1000 kcal.

The nutritional composition may further comprise glutamine and/or arginine, although the nutritional composition of the present invention allows the body to sustain its metabolic needs for arginine and glutamine without relying on exogenous direct supply for these two amino acids.

Suitably, the nutritional composition may comprise:
(i) glutamine in an amount of about 20 g/1000 kcal or less, about 10 g/1000 kcal or less, about 5 g/1000 kcal or less, or about 2.5 g/1000 kcal or less,
(ii) arginine in an amount of about 15 g/1000 kcal or less, about 7.5 g/1000 kcal or less, about 4 g/1000 kcal or less, about 2.0 g/1000 kcal or less, or about 1.0 g/1000 kcal or less.

Suitably, the nutritional composition may comprise:
(i) glutamine in an amount of about 20% or less, about 15% or less, about 10% or less, or about 5% or less, as a percentage of total protein; and/or
(ii) arginine in an amount of about 15% or less, about 7.5% or less, about 4% or less, about 3% or less, or about 2% or less, as a percentage of total protein.

In some embodiments, the nutritional composition does not comprise added glutamine and/or arginine. The term “added glutamine and/or arginine” does not include glutamine and/or arginine present in minor amounts as natural constituents of other ingredients of the nutritional composition (e.g. the protein source). For example, in some embodiments, other than the content of glutamine and/arginine provided by any protein source, the nutritional composition does not comprise further glutamine and/or arginine (e.g. added in the form of a free amino acid, or as a salt thereof).

In some embodiments, the nutritional composition does not comprise glutamine and/or arginine as a free amino acid, or as a salt thereof. In some embodiments, the nutritional composition is substantially devoid of glutamine and/or arginine. In some embodiments, the nutritional does not comprise glutamine and/or arginine.

### Protein, Carbohydrate, and Lipids

The nutritional composition may include all the nutrients required to maintain health. The nutritional composition may contain a protein source, a carbohydrate source and/or a lipid source.

The protein may be present in the nutritional composition in any suitable amount. For example, the protein content of the nutritional composition may be from about 30 g/L to about 200 g/L, from about 40 g/L to about 150 g/L, or from about 50 g/L to about 120 g/L.

The protein source may be any protein source which is suitable for use in a nutritional composition. Protein sources based on, for example, milk proteins, whey, casein and mixtures thereof may be used, as well as protein sources based on soy. Keratin protein sources may also be used. Suitable sources of keratin protein include sheep wool, egg shell membrane and poultry feathers. As far as whey proteins are concerned, the protein source may be based on acid whey or sweet whey or mixtures thereof.

The proteins may be intact or hydrolysed or a mixture of intact and hydrolysed proteins. By the term “intact” is meant that the main part of the proteins are intact, i.e. the molecular structure is not altered, for example at least 80%, at least 85%, at least 90%, at least 95%, at least 98%, or 100% of the proteins are not altered.

The term “hydrolysed” means in the context of the present invention a protein which has been hydrolysed or broken down into its component amino acids. The proteins may be either fully or partially hydrolysed. The degree of hydrolysis (DH) of the protein can be between 2 and 20, or between 8 and 40, or between 20 and 60 or between 20 and 80 or more than 10, 20, 40, 60, 80 or 90. Suitably, at least 70%, at least 80%, at least 85%, at least 90%, at least 95%, at least 98%, or 100% of the proteins may be hydrolysed. If hydrolysed proteins are required, the hydrolysis process may be carried out as desired and as is known in the art. For example, whey protein hydrolysates may be prepared by enzymatically hydrolysing the whey fraction in one or more steps.

The lipids (fat) may be present in the nutritional composition in any suitable amount. For example, the fat content of the nutritional composition may be from about 10 g/L to about 200 g/L, from about 15 g/L to about 150 g/L, or from about 20 g/L to about 100 g/L.

Example fats for use in the nutritional composition of the invention include sunflower oil, low erucic acid rapeseed oil, safflower oil, canola oil, olive oil, coconut oil, palm kernel oil, soybean oil, fish oil, palm oleic, high oleic sunflower oil and high oleic safflower oil, and microbial fermentation oil containing long chain, polyunsaturated fatty acids. The fat may also be in the form of fractions derived from these oils, such as palm olein, medium chain triglycerides (MCT) and esters of fatty acids such as arachidonic acid, linoleic acid, palmitic acid, stearic acid, docosahexaenoic acid, linolenic acid, oleic acid, lauric acid, capric acid, caprylic acid, caproic acid, and the like. Further example fats include structured lipids (i.e. lipids that are modified chemically or enzymatically in order to change their structure).

Long chain polyunsaturated fatty acids, such as dihomo-γ-linolenic acid, arachidonic acid (ARA), eicosapentaenoic acid and docosahexaenoic acid (DHA), may also be added. The essential fatty acids linoleic and α-linolenic acid may also be added, as well small amounts of oils containing high quantities of preformed arachidonic acid and docosahexaenoic acid such as fish oils or microbial oils.

The carbohydrate may be present in the nutritional composition in any suitable amount. For example, the carbohydrate content of the nutritional composition may be from about 30 g/L to about 300 g/L, from about 40 g/L to about 250 g/L, from about 50 g/L to about 200 g/L, or from about 100 g/L to 200 g/L.

The carbohydrate source may be any carbohydrate source which is suitable for use in a nutritional composition. Some suitable carbohydrate sources include lactose, sucrose, saccharose, maltodextrin, starch and mixtures thereof.

### Other Nutrients

The nutritional composition of the invention may also contain any suitable vitamins and minerals.

For example, the nutritional composition of the invention may contain all vitamins and minerals understood to be essential in the daily diet and in nutritionally significant amounts. Minimum requirements have been established for certain vitamins and minerals. Examples of minerals, vitamins and other nutrients optionally present in the nutritional composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

The nutritional composition of the invention may contain emulsifiers and stabilisers such as soy, lecithin, citric acid esters of mono- and diglycerides, and the like.

The nutritional composition of the invention may also contain one or more carotenoid.

The nutritional composition of the invention may also contain other substances which may have a beneficial effect such as lactoferrin, osteopontin, TGFbeta, sIgA, glutamine, nucleotides, nucleosides, and the like.

The nutritional composition of the invention can further comprise at least one non-digestible oligosaccharide (e.g. prebiotics). Examples of prebiotics may be fructooligosaccharides and galactooligosaccharides.

The nutritional composition of the present invention can further comprise at least one probiotic. The term “probiotic” may refer to microbial cell preparations or components of microbial cells with beneficial effects on the health or well-being of the host. In particular, probiotics may improve gut barrier function. Examples of probiotic micro-organisms for use in the nutritional composition of the present invention include yeasts, such as Saccharomyces, Debaromyces, Candida, Pichia and Torulopsis; and bacteria, such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus and Staphylococcus xylosus.

### Energy

The nutritional composition may provide a suitable amount of energy to maintain health. Suitably, the nutritional composition may comprise from about 500 kcal/L to about 3000 kcal/L, from about 750 kcal/L to about 2500 kcal/L, or from about 1000 kcal/L to about 2000 kcal/L.

### Supplement or Fortifier

In one aspect, the present invention provides a supplement or fortifier comprising threonine, serine, proline, leucine, and cysteine.

A “supplement” or “dietary supplement” may be used to complement the nutrition of an individual (it is typically used as such but it might also be added to a nutritional composition). The term “fortifier” may refer to liquid or solid compositions suitable for mixing with nutritional compositions.

The supplement or fortifier may be in the form of for example sticks, tablets, capsules, pastilles or a liquid. In some embodiments, the supplement or fortifier is in the form of a stick.

The supplement or fortifier may contain an organic or inorganic carrier material suitable for enteral or oral administration (e.g. maltodextrin) as well as vitamins, minerals trace elements and other micronutrients in accordance with the recommendations of Government bodies such as the USRDA. The supplement or fortifier may be provided in the form of unit doses.

The supplement of fortifier may comprise any suitable amount of threonine, serine, proline, leucine, and cysteine. For example, suitable weight ratios are described in the section entitled “Amino acid composition” and suitable doses are described in the section entitled “Use as a medicament”.

The supplement of fortifier may be 5-times concentrated, 10-times concentrated, 15-times concentrated, 20-times concentrated, 25-times concentrated, 30-times concentrated, 35-times concentrated, 40-times concentrated, 45-times concentrated, 50-times concentrated, 60-times concentrated, 70-times concentrated, 80-times concentrated, 90-times concentrated, or 100-times concentrated, compared to the desired final concentration in the nutritional composition. Suitable final concentrations are described in the section entitled “Nutritional composition”.

In some embodiments, the supplement or fortifier does not comprise added glutamine and/or arginine. In some embodiments, the supplement or fortifier does not comprise glutamine and/or arginine as a free amino acid, or as a salt thereof. In some embodiments, the supplement or fortifier is substantially devoid of glutamine and/or arginine. In some embodiments, the supplement or fortifier does not comprise glutamine and/or arginine.

### Use as a Medicament

As described above, the present inventors have shown that a combination of threonine, serine, proline, leucine, and cysteine can contribute to a favourable clinical outcome in critically ill patients.

In one aspect, the present invention provides a combination of threonine, serine, proline, leucine, and cysteine for use as a medicament.

In another aspect, the present invention provides a combination of threonine, serine, proline, leucine, and cysteine for the manufacture of a medicament.

In another aspect, the present invention provides a method of treatment comprising administering a combination of threonine, serine, proline, leucine, and cysteine.

The combination of threonine, serine, proline, leucine, and cysteine may be administered using any suitable method. Suitably, the combination may be administered in the form of an amino acid composition, nutritional composition, supplement, or fortifier according to the present invention.

In one aspect, the present invention provides an amino acid composition, nutritional composition, supplement, or fortifier according to the present invention for use as a medicament.

In another aspect, the present invention provides use of an amino acid composition, nutritional composition, supplement, or fortifier according to the present invention for the manufacture of a medicament.

In another aspect, the present invention provides a method of treatment comprising administering an amino acid composition, nutritional composition, supplement, or fortifier according to the present invention.

### Subject

The amino acids (e.g. in the form of an amino acid composition, nutritional composition, supplement, or fortifier) may be administered to any suitable subject. The subject may be a human subject.

The subject may have a condition which prevents eating a regular diet by mouth, but in which their gastrointestinal tract is still able to function. For example, the subject may have a critical or serious illness, dementia, mechanical obstruction or dysmotility, or may have had gastrointestinal surgery.

A “serious illness” may refer to health conditions that carry a high risk of mortality. A “critical illness” may refer to a life-threatening medical or surgical condition usually requiring intensive care unit (ICU)-level care that includes, but is not limited to, trauma, surgery, sepsis, shock, acute respiratory failure, pancreatitis and severe burns.

In some embodiments, the subject has a critical illness. In some embodiments, the critical illness is selected from one or more of: sepsis, acute respiratory failure, trauma, surgery, shock, and severe burns. In some embodiments, the critical illness is sepsis or acute respiratory failure.

In some embodiments, the subject has mucositis associated with cancer, inflammatory bowel disease, distorted gut barrier, intestinal mucosal lesions or short-bowel syndrome.

In some embodiments, the subject is undergoing rehabilitation after surgery.

In some embodiments, the subject is a premature child.

“Sepsis” is a life-threatening organ dysfunction caused by a dysregulated host response to infection. Sepsis may be diagnosed according to the Third International Consensus Definitions for Sepsis and Septic Shock (Sepsis-3) (see Singer, M., et al., 2016. Jama, 315 (8), pp. 801-810).

“Acute respiratory failure” includes acute respiratory distress syndrome (ARDS) which is a type of respiratory failure characterized by rapid onset of widespread inflammation in the lungs. ARDS may be diagnosed as described in Fan, E., et al., 2018. Jama, 319 (7), pp. 698-710.

In some embodiments, the subject is a critically ill patient. A “critically ill patient” may refer to a group of patients with a life-threatening medical or surgical status who may need treatment in an ICU.

The subject may be any age. For example, the subject may be a child or an adult. The term “child” may refer to a subject aged under 18 years. The term “adult” may refer to a subject aged 18 years or older. In some embodiments, the subject is an adult. In some embodiments, the subject is a child.

### Routes of Administration

The amino acids (e.g. in the form of an amino acid composition, nutritional composition, supplement, or fortifier) may be administered by any suitable route, for example by enteral, oral, or parenteral administration.

In some embodiments, the amino acids (e.g. in the form of an amino acid composition, nutritional composition, supplement, or fortifier) are administered enterally. For example, the amino acids (e.g. in the form of an amino acid composition, nutritional composition, supplement, or fortifier) may be administered by tube feeding. Any suitable feeding tube can be used, for example a nasogastric feeding tube, a nasojejunal feeding tube, a gastric feeding tube, a gastrojejunal feeding tube, or a jejunal feeding tube. Suitably, the amino acids (e.g. in the form of an amino acid composition, nutritional composition, supplement, or fortifier) may be administered enterally in the form of an enteral nutritional composition.

### Dose Administered

The amino acids (e.g. in the form of an amino acid composition, nutritional composition, supplement, or fortifier) may be administered in any suitable dose. For example, when administered as an enteral nutritional composition, the subject may be administered a dose based on recommended guidelines (e.g. McClave S A, et al. JPEN J Parenter Enteral Nutr 2016; 40 (2): 159-211), e.g. a dose corresponding to about 25 kcal/kg/day to about 40 kcal/kg/day.

The subject may be administered each of threonine, serine, proline, leucine, and cysteine in an amount of about 0.01 g/kg/day or more, about 0.02 g/kg/day or more, about 0.03 g/kg/day or more, about 0.04 g/kg/day or more, about 0.05 g/kg/day or more, or about 0.1 g/kg/day or more.

The subject may be administered each of threonine, serine, proline, leucine, and cysteine in an amount of about 1 g/kg/day or less, about 0.5 g/kg/day or less, about 0.4 g/kg/day or less, about 0.3 g/kg/day or less, about 0.2 g/kg/day or less, or about 0.1 g/kg/day or less.

The subject may be administered each of threonine, serine, proline, leucine, and cysteine in an amount of from about 0.01 g/kg/day to about 1 g/kg/day, from about 0.02 g/kg/day to 0.5 g/kg/day, from about 0.03 g/kg/day to about 0.4 g/kg/day, from about 0.04 g/kg/day to about 0.3 g/kg/day, or from about 0.05 g/kg/day or more to about 0.2 g/kg/day.

Suitably, the subject may be administered threonine in an amount of about 0.06 g/kg/day or more, about 0.08 g/kg/day or more, or about 0.1 g/kg/day.

Suitably, the subject may be administered threonine in an amount of about 0.5 g/kg/day or less, about 0.4 g/kg/day or less, or about 0.3 g/kg/day or less.

Suitably, the subject may be administered threonine in an amount of about 0.06 g/kg/day to about 0.5 g/kg/day, from about 0.08 g/kg/day to about 0.4 g/kg/day, or from about 0.1 g/kg/day to about 0.3 g/kg/day.

Suitably, the subject may be administered serine in an amount of about 0.04 g/kg/day or more, about 0.06 g/kg/day or more, or about 0.08 g/kg/day or more.

Suitably, the subject may be administered serine in an amount of about 0.4 g/kg/day or less, about 0.3 g/kg/day or less, or about 0.2 g/kg/day or less.

Suitably, the subject may be administered serine in an amount of from about 0.04 g/kg/day to about 0.4 g/kg/day, from about 0.06 g/kg/day to about 0.3 g/kg/day, or from about 0.08 g/kg/day to about 0.2 g/kg/day.

Suitably, the subject may be administered proline in an amount of about 0.02 g/kg/day or more, about 0.03 g/kg/day or more, or about 0.04 g/kg/day or more.

Suitably, the subject may be administered proline in an amount of about 0.4 g/kg/day or less, about 0.3 g/kg/day or less, or about 0.2 g/kg/day or less.

Suitably, the subject may be administered proline in an amount of about 0.02 g/kg/day to about 0.4 g/kg/day, from about 0.03 g/kg/day to about 0.3 g/kg/day, or from about 0.04 g/kg/day to about 0.2 g/kg/day.

Suitably, the subject may be administered leucine in an amount of about 0.04 g/kg/day or more, about 0.06 g/kg/day or more, or about 0.08 g/kg/day or more.

Suitably, the subject may be administered leucine in an amount of about 0.5 g/kg/day or less, about 0.4 g/kg/day or less, or about 0.3 g/kg/day or less.

Suitably, the subject may be administered leucine in an amount of about 0.04 g/kg/day to about 0.5 g/kg/day, from about 0.06 g/kg/day to about 0.4 g/kg/day, or from about 0.08 g/kg/day to about 0.3 g/kg/day.

Suitably, the subject may be administered cysteine in an amount of about 0.02 g/kg/day or more, about 0.04 g/kg/day or more, or about 0.06 g/kg/day or more.

Suitably, the subject may be administered cysteine in an amount of about 0.4 g/kg/day or less, about 0.3 g/kg/day or less, or about 0.2 g/kg/day or less.

Suitably, the subject may be administered cysteine in an amount of about 0.02 g/kg/day to about 0.4 g/kg/day, from about 0.04 g/kg/day to about 0.3 g/kg/day, or from about 0.06 g/kg/day to about 0.2 g/kg/day.

In some embodiments, the subject is administered:
(i) threonine in an amount of from about 0.06 g/kg/day to about 0.5 g/kg/day, from about 0.08 g/kg/day to about 0.4 g/kg/day, or from about 0.1 g/kg/day to about 0.3 g/kg/day;
(ii) serine in an amount of from about 0.04 g/kg/day to about 0.4 g/kg/day, from about 0.06 g/kg/day to about 0.3 g/kg/day, or from about 0.08 g/kg/day to about 0.2 g/kg/day;
(iii) proline in an amount of from about 0.02 g/kg/day to about 0.4 g/kg/day, from about 0.03 g/kg/day to about 0.3 g/kg/day, or from about 0.04 g/kg/day to about 0.2 g/kg/day;
(iv) leucine in an amount of from about 0.04 g/kg/day to about 0.5 g/kg/day, from about 0.06 g/kg/day to about 0.4 g/kg/day, or from about 0.08 g/kg/day to about 0.3 g/kg/day; and
(v) cysteine in an amount of from about 0.02 g/kg/day to about 0.4 g/kg/day, from about 0.04 g/kg/day to about 0.3 g/kg/day, or from about 0.06 g/kg/day to about 0.2 g/kg/day.

Suitable doses of each of threonine, serine, proline, leucine, and cysteine may also be based on the amounts of each amino acid present in the amino acid composition, nutritional composition, supplement or fortifier. This will be the case, for example, if the amino acid composition, nutritional composition, supplement or fortifier provides the subject with their only source threonine, serine, proline, leucine, and cysteine.

For example, if the amino acid composition comprises each of threonine, serine, proline, leucine, and cysteine in a weight ratio (threonine:serine:proline:leucine:cysteine) of about 0.1-10:0.1-10:0.1-10:0.1-10:0.1-10, or about 0.2-8:0.2-8:0.2-8:0.2-8:0.2-8, or about 0.3-6:0.3-6:0.3-6:0.3-6:0.3-6, or about 0.4-5:0.4-5:0.4-5:0.4-5:0.4-5, or about 0.5-3:0.5-3:0.5-3:0.5-3:0.5-3, the subject may be administered each of threonine, serine, proline, leucine, and cysteine in the same weight ratio.

For example, if the nutritional composition comprises each of each of threonine, serine, proline, leucine, and cysteine in an amount of about 1% or more, about 2% or more, about 3% or more, about 4% or more, or about 5% or more as a percentage of total protein, the subject may be administered each of threonine, serine, proline, leucine, and cysteine in the same amount.

### Other Amino Acids

The subject may also be administered glutamine and/or arginine, although the present invention allows the body to sustain its metabolic needs for arginine and glutamine without relying on exogenous direct supply for these two amino acids.

Suitably, the subject may be administered:
(i) glutamine in an amount of about 0.1 g/kg/day or less, about 0.05 g/kg/day or less, about 0.04 g/kg/day or less, about 0.03 g/kg/day or less, about 0.02 g/kg/day or less, about 0.01 g/kg/day or less, or less than about 0.01 g/kg/day; and/or
(ii) arginine in an amount of about 0.1 g/kg/day or less, about 0.05 g/kg/day or less, about 0.04 g/kg/day or less, about 0.03 g/kg/day or less, about 0.02 g/kg/day or less, about 0.01 g/kg/day or less, or less than about 0.01 g/kg/day.

Suitably, the subject may be administered:
(i) glutamine in an amount of about 4% or less, about 3% or less, about 2% or less, about 1% or less, or about 0.5% or less as a percentage of total protein; and/or
(ii) arginine in an amount of about 4% or less, about 3% or less, about 2% or less, about 1% or less, or about 0.5% or less as a percentage of total protein.

In some embodiments, the subject is not administered added glutamine and/or arginine. In some embodiments, the subject is not administered glutamine and/or arginine as a free amino acid, or as a salt thereof.

### Clinical Outcome

As described above, the inventors found that administration of threonine, serine, proline, leucine, and cysteine resulted in favorable clinical outcomes compared to an isocaloric enteral feed, including a shorter time of ventilation and shorter stay in ICU, as well normalization of cholesterol level, which is a positive prognostic in ICU patients.

In some embodiments, the subject exhibits a reduced ventilation time, shorter stay in ICU, and/or increased total cholesterol. The subject may exhibit an increase in total cholesterol for at least 7 days post-admission, for at least 14 days post-admission, for at least 21 days post admission, or for at least 60 days post-admission. The reduced ventilation time, shorter stay in ICU, and/or increased total cholesterol may be statistically significant compared to a subject who is not administered the amino acids (e.g. a subject who is administered an isocaloric composition).

### Methods of Promoting Gut Healing

As described above, the inventors have shown that a combination of threonine, serine, proline, leucine, and cysteine can speed up the gut healing process in critically ill patients.

In one aspect, the present invention provides a combination of threonine, serine, proline, leucine, and cysteine for use in promoting gut healing in a subject.

In another aspect, the present invention provides a combination of threonine, serine, proline, leucine, and cysteine for the manufacture of a medicament for promoting gut healing in a subject.

In another aspect, the present invention provides a method of promoting gut healing in a subject comprising administering a combination of threonine, serine, proline, leucine, and cysteine.

In another aspect, the present invention provides an amino acid composition, nutritional composition, supplement, or fortifier according to the present invention for use in promoting gut healing in a subject.

In another aspect, the present invention provides use of an amino acid composition, nutritional composition, supplement, or fortifier according to the present invention for the manufacture of a medicament for promoting gut healing in a subject.

In another aspect, the present invention provides a method of promoting gut healing in a subject comprising administering an amino acid composition, nutritional composition, supplement, or fortifier according to the present invention.

### Gut Barrier and Function

The amino acids (e.g. in the form of an amino acid composition, nutritional composition, supplement, or fortifier) may promote gut healing by speeding up or promoting recovery of gut barrier structure and/or function.

The “gut barrier” or “intestinal barrier” is a semipermeable structure that allows the uptake of essential nutrients and immune sensing, while being restrictive against pathogenic molecules and bacteria. The function of the intestinal barrier can be compromised through severe structural damage of the mucosa, or more subtle changes in the regulating components of the barrier. Intestinal barrier defects have been associated with a broad range of diseases. (Vancamelbeke, M. and Vermeire, S., 2017. Expert review of gastroenterology & hepatology, 11 (9), pp. 821-834).

Gastrointestinal dysfunction in critically ill patients is common and associated with a poor prognosis. Many factors can cause critically ill patients to lose gut barrier function by a mechanism of enterocyte damage, including, for example, small bowel ischemia or hypoxia, sepsis, systemic inflammatory response syndrome, or absence of enteral feeding (Piton, G. and Capellier, G., 2016. Current opinion in critical care, 22 (2), pp. 152-160). Gut barrier failure in critically ill patients is associated with bacterial translocation, systemic inflammation, and the development of multiple organ dysfunction syndrome.

In one aspect, the present invention provides an amino acid composition, nutritional composition, supplement, or fortifier according to the present invention for use in promoting recovery of gut barrier structure and/or function in a subject.

In another aspect, the present invention provides use of an amino acid composition, nutritional composition, supplement, or fortifier according to the present invention for the manufacture of a medicament for promoting recovery of gut barrier structure and/or function in a subject.

In another aspect, the present invention provides a method of promoting recovery of gut barrier structure and/or function in a subject comprising administering an amino acid composition, nutritional composition, supplement, or fortifier according to the present invention.

### Functional Enterocyte Mass

Critically ill patients with shock have an acute reduction of enterocyte mass and reduced gut citrulline synthesis, leading to a low plasma citrulline concentration. Acute intestinal failure can be defined as an acute reduction of enterocyte mass and/or acute dysfunction of enterocytes, associated or not with loss of gut barrier function (Piton, G., et al., 2011. Intensive care medicine, 37 (6), pp. 911-917).

As described above, the inventors have shown that a combination of threonine, serine, proline, leucine, and cysteine was able to boost recovery of metabolically active enterocyte mass, as shown by an increase in plasma citrulline concentrations.

In one aspect, the present invention provides an amino acid composition, nutritional composition, supplement, or fortifier according to the present invention for use in promoting recovery of enterocyte mass in a subject.

In another aspect, the present invention provides use of an amino acid composition, nutritional composition, supplement, or fortifier according to the present invention for the manufacture of a medicament for promoting recovery of enterocyte mass in a subject.

In another aspect, the present invention provides a method of promoting recovery of enterocyte mass in a subject comprising administering an amino acid composition, nutritional composition, supplement, or fortifier according to the present invention.

In some embodiments, the subject exhibits an increase in plasma concentration of citrulline. The subject may exhibit an increase in plasma concentration of citrulline for at least 7 days post-admission, for at least 14 days post-admission, for at least 21 days post admission, or for at least 60 days post-admission. The increase in plasma concentration of citrulline may be statistically significant compared to a subject who is not administered the amino acids (e.g. a subject who is administered an isocaloric composition).

### Glutamine Deficiency and Arginine Deficiency

The subject may be at risk of or may have a glutamine deficiency and/or an arginine deficiency. Such subjects may be identified by any suitable method. For example, systemic arginine availability may be estimated by calculation of the ratios of arginine to its enzymatic products (citrulline and ornithine) or arginine to its endogenous metabolic inhibitors (asymmetric and symmetric dimethylarginines) (see e.g. Costa, B. P., et al., 2016. Nutrition & metabolism, 13 (1), pp. 1-9). Plasma glutamine levels and high CRP values may indicated potential glutamine deficiency (see e.g. Nienaber, A., et al., 2015. Nutrition journal, 15 (1), pp. 1-9).

Glutamine deficiency and arginine deficiency are described further below in the section entitled “Methods of preventing and/or reducing glutamine deficiency and/or arginine deficiency”.

Methods of Preventing and/or Reducing Muscle Loss

As described above, the inventors have surprisingly shown that a combination of threonine, serine, proline, leucine, and cysteine can decrease muscle protein breakdown in critically ill patients and was associated with improved skeletal muscle and diaphragm functions.

In one aspect, the present invention provides a combination of threonine, serine, proline, leucine, and cysteine for use in preventing and/or reducing muscle loss in a subject.

In another aspect, the present invention provides a combination of threonine, serine, proline, leucine, and cysteine for the manufacture of a medicament for preventing and/or reducing muscle loss in a subject.

In another aspect, the present invention provides a method of preventing and/or reducing muscle loss in a subject comprising administering a combination of threonine, serine, proline, leucine, and cysteine.

In another aspect, the present invention provides an amino acid composition, nutritional composition, supplement, or fortifier according to the present invention for use in preventing and/or reducing muscle loss in a subject.

In another aspect, the present invention provides use of an amino acid composition, nutritional composition, supplement, or fortifier according to the present invention for the manufacture of a medicament for preventing and/or reducing muscle loss in a subject.

In another aspect, the present invention provides a method of preventing and/or reducing muscle loss in a subject comprising administering an amino acid composition, nutritional composition, supplement, or fortifier according to the present invention.

The term “muscle loss” may refer to any condition associated with muscle protein breakdown and may include reduced muscle strength, mass, and/or function. For example, the term “muscle loss” may include muscle wasting, muscle atrophy, muscle weakness and/or loss of muscle function. In some embodiments, amino acids (e.g. in the form of an amino acid composition, nutritional composition, supplement, or fortifier) may be used to prevent and/or reduce muscle wasting, muscle atrophy, muscle weakness, and/or loss of muscle function.

The amino acids (e.g. in the form of an amino acid composition, nutritional composition, supplement, or fortifier) may be used to prevent and/or treat any muscle loss condition associated with critical illness (see e.g. Schefold, J. C., et al., 2020. Journal of cachexia, sarcopenia and muscle, 11 (6), pp. 1399-1412). Muscle loss conditions associated with critical illness may include: intensive care unit-acquired weakness (ICUAW), critical illness polyneuropathy (CIP), critical illness myopathy (CIM), muscle wasting, ventilator-induced diaphragmatic dysfunction (VIDD), and dysphagia.

The amino acids (e.g. in the form of an amino acid composition, nutritional composition, supplement, or fortifier) may be used to prevent and/or treat intensive care unit-acquired weakness (ICUAW). A substantial number of patients admitted to the ICU because of an acute illness, complicated surgery, severe trauma, or burn injury will develop a de novo form of muscle weakness during the ICU stay that is referred to as “intensive care unit acquired weakness” (ICUAW). This ICUAW evoked by critical illness can be due to axonal neuropathy, primary myopathy, or both. ICUAW may be diagnosed in awake and cooperative patients by bedside manual testing of muscle strength and the severity is scored by the Medical Research Council sum score (Hermans, G. and Van den Berghe, G., 2015. Critical care, 19 (1), pp. 1-9).

The amino acids (e.g. in the form of an amino acid composition, nutritional composition, supplement, or fortifier) may be used to prevent and/or treat muscle wasting. Muscular wasting may constitute a separate disease entity. Whereas ICUAW is often associated with muscle wasting, muscle wasting does not per se imply the presence of a neuromuscular disorder (Schefold, J. C., et al., 2010. Journal of cachexia, sarcopenia and muscle, 1 (2), pp. 147-157).

The amino acids (e.g. in the form of an amino acid composition, nutritional composition, supplement, or fortifier) may be used to prevent and/or treat critical illness polyneuropathy (CIP) and/or critical illness myopathy (CIM). Critical illness polyneuropathy (CIP) is a frequent complication of critical illness, acutely and primarily affecting the motor and sensory axons. Improvement of diagnostics later on revealed that muscle may be primarily involved, which is called myopathy in critical illness or critical illness myopathy (CIM). (Hermans, G., et al., 2008. Critical care, 12 (6), pp. 1-9).

The amino acids (e.g. in the form of an amino acid composition, nutritional composition, supplement, or fortifier) may be used to prevent and/or treat ventilator-induced diaphragmatic dysfunction (VIDD). Diaphragmatic dysfunction is common in mechanically ventilated patients and is a likely cause of weaning failure. VIDD may refer to changes in diaphragmatic function that arise from alterations outside of the central or peripheral nervous systems (Petrof, B. J., et al., 2010. Current opinion in critical care, 16 (1), pp. 19-25).

The amino acids (e.g. in the form of an amino acid composition, nutritional composition, supplement, or fortifier) may prevent and/or reduce muscle loss by promoting gut healing, as described above in the section entitled “Methods of promoting gut healing”. Suitably, the amino acids (e.g. in the form of an amino acid composition, nutritional composition, supplement, or fortifier) may prevent and/or reduce muscle loss by promoting recovery of gut barrier structure and/or function. Suitably, the amino acids (e.g. in the form of an amino acid composition, nutritional composition, supplement, or fortifier) may prevent and/or reduce muscle loss by promoting recovery of enterocyte mass.

### Skeletal Muscle Mass and Strength

A large majority of patients admitted to the intensive care unit (ICU) after the very acute phase of a critical illness exhibit major defects in skeletal-muscle strength (weakness) and mass (wasting) (Maffiuletti, N. A., et al., 2013. BMC medicine, 11 (1), pp. 1-10). The amino acids (e.g. in the form of an amino acid composition, nutritional composition, supplement, or fortifier) may prevent or reduce the loss of skeletal muscle mass and/or strength.

In one aspect, the present invention provides an amino acid composition, nutritional composition, supplement, or fortifier according to the present invention for use in preventing and/or reducing loss of skeletal muscle mass and/or strength in a subject.

In another aspect, the present invention provides use of an amino acid composition, nutritional composition, supplement, or fortifier according to the present invention for the manufacture of a medicament for preventing and/or reducing loss of skeletal muscle mass and/or strength in a subject.

In another aspect, the present invention provides a method of preventing and/or reducing loss of skeletal muscle mass and/or strength in a subject comprising administering an amino acid composition, nutritional composition, supplement, or fortifier according to the present invention.

The amino acids (e.g. in the form of an amino acid composition, nutritional composition, supplement, or fortifier) may increase the skeletal muscle mass (e.g. the volume of the anterior compartment of the quadriceps). The subject may have an increased skeletal muscle mass (e.g. the volume of the anterior compartment of the quadriceps) at least 7 days post-admission, at least 14 days post-admission, at least 21 days post admission, or at least 60 days post-admission. The increase in skeletal muscle mass (e.g. the volume of the anterior compartment of the quadriceps) may be statistically significant compared to a subject who is not administered the amino acids (e.g. a subject who is administered an isocaloric composition).

The amino acids (e.g. in the form of an amino acid composition, nutritional composition, supplement, or fortifier) may increase the skeletal muscle strength (e.g. quadriceps strength). The subject may have an increased skeletal muscle strength (e.g. quadriceps strength) at least 7 days post-admission, at least 14 days post-admission, at least 21 days post admission, or at least 60 days post-admission. The increase in skeletal muscle strength (e.g. quadriceps strength) may be statistically significant compared to a subject who is not administered the amino acids (e.g. a subject who is administered an isocaloric composition).

### Diaphragm Muscle Strength

Studies indicate that mechanically ventilated patients develop significant diaphragm muscle weakness (Supinski, G. S. and Callahan, L. A., 2013. Critical care, 17 (3), pp. 1-17). The amino acids (e.g. in the form of an amino acid composition, nutritional composition, supplement, or fortifier) may prevent and/or reduce diaphragm muscle weakness.

In one aspect, the present invention provides an amino acid composition, nutritional composition, supplement, or fortifier according to the present invention for use in preventing and/or reducing diaphragm muscle weakness in a subject.

In another aspect, the present invention provides use of an amino acid composition, nutritional composition, supplement, or fortifier according to the present invention for the manufacture of a medicament for preventing and/or reducing diaphragm muscle weakness in a subject.

In another aspect, the present invention provides a method of preventing and/or reducing diaphragm muscle weakness in a subject comprising administering an amino acid composition, nutritional composition, supplement, or fortifier according to the present invention.

The amino acids (e.g. in the form of an amino acid composition, nutritional composition, supplement, or fortifier) may increase the diaphragm muscle strength. The subject may have an increased diaphragm muscle strength at least 7 days post-admission, at least 14 days post-admission, at least 21 days post admission, or at least 60 days post-admission. The increase in diaphragm muscle strength may be statistically significant compared to a subject who is not administered the amino acids (e.g. a subject who is administered an isocaloric composition).

Methods of Preventing and/or Reducing Glutamine Deficiency and/or Arginine Deficiency

As described above, the inventors have shown that a combination of threonine, serine, proline, leucine, and cysteine can protect body pools of glutamine and arginine without enriching the enteral feed directly with these two amino acids.

Several nonessential amino acids may become essential under conditions of stress and catabolic states when the capacity of endogenous amino acid synthesis is exceeded. Arginine and glutamine are two such conditionally essential amino acids. Glutamine, an arginine precursor, is one of the most abundant amino acids in the body and can become deficient in critically ill patients (Morris, C. R., et al., 2017. Nutrition in Clinical Practice, 32, pp. 30S-47S). Arginine deficiency in critical illness is associated with impairments in microcirculatory blood flow, impaired wound healing, and T-cell dysfunction (Patel, J. J., et al., 2016. Nutrition in Clinical Practice, 31 (4), pp. 438-444).

Direct supply of glutamine or arginine is, however, difficult for technical reasons (glutamine is not stable in ready-to-feed enteral nutrition products) and controversial for safety reasons. Direct supply of glutamine or arginine has been previously shown to induce untoward effects including death in this critically ill patient population (Heyland, D., et al., 2013. New England Journal of Medicine, 368 (16), pp. 1489-1497; and Rosenthal, M. D., et al., 2016. Int J Crit Care Emerg Med, 2, p. 017). The reasons for these safety issues are still not well understood, but may be due to temporary high peak values in plasma causing pharmacological effects such as vasodilation and hypovolemic shock.

A combination of threonine, serine, proline, leucine, and cysteine is able to cover at the same time amino acid requirements of the splanchnic area and the muscle, which may prevent a deficiency of glutamine and/or arginine. Consequently, the combination of threonine, serine, proline, leucine, and cysteine allows the body to sustain its metabolic needs for arginine and glutamine without relying on exogenous direct supply for these two amino acids, avoiding the difficulties discussed above.

In one aspect, the present invention provides a combination of threonine, serine, proline, leucine, and cysteine for use in preventing and/or reducing glutamine deficiency and/or arginine deficiency in a subject.

In another aspect, the present invention provides a combination of threonine, serine, proline, leucine, and cysteine for the manufacture of a medicament for preventing and/or reducing glutamine deficiency and/or arginine deficiency in a subject.

In another aspect, the present invention provides a method of preventing and/or reducing glutamine deficiency and/or arginine deficiency in a subject comprising administering a combination of threonine, serine, proline, leucine, and cysteine.

In another aspect, the present invention provides an amino acid composition, nutritional composition, supplement, or fortifier according to the present invention for use in preventing and/or reducing glutamine deficiency and/or arginine deficiency in a subject.

In another aspect, the present invention provides use of an amino acid composition, nutritional composition, supplement, or fortifier according to the present invention for the manufacture of a medicament for preventing and/or reducing glutamine deficiency and/or arginine deficiency in a subject.

In another aspect, the present invention provides a method of preventing and/or reducing glutamine deficiency and/or arginine deficiency in a subject comprising administering an amino acid composition, nutritional composition, supplement, or fortifier according to the present invention.

The amino acids (e.g. in the form of an amino acid composition, nutritional composition, supplement, or fortifier) may prevent and/or reduce glutamine deficiency and/or arginine deficiency by promoting gut healing, as described above in the section entitled “Methods of promoting gut healing”. Suitably, the amino acids (e.g. in the form of an amino acid composition, nutritional composition, supplement, or fortifier) may prevent and/or reduce glutamine deficiency and/or arginine deficiency by promoting recovery of gut barrier structure and/or function. Suitably, the amino acids (e.g. in the form of an amino acid composition, nutritional composition, supplement, or fortifier) may prevent and/or reduce glutamine deficiency and/or arginine deficiency by promoting recovery of enterocyte mass.

### Methods of Manufacture

In one aspect, the present invention provides a method of producing an amino acid composition, nutritional composition, supplement, or fortifier according to the present invention.

The method may comprise: (i) providing a base amino acid composition, nutritional composition, supplement, or fortifier; and (ii) adding threonine, serine, proline, leucine, and cysteine to the base an amino acid composition, nutritional composition, supplement, or fortifier to provide an amino acid composition, nutritional composition, supplement, or fortifier according to the present invention.

In another aspect, the present invention provides a method of producing an enteral nutritional composition according to the present invention. The method may comprise: (i) providing a base enteral nutritional composition; and (ii) adding threonine, serine, proline, leucine, and cysteine to the base enteral nutritional composition to provide an enteral nutritional composition according to the present invention.

The amino acid composition, nutritional composition, supplement, or fortifier of the present invention may be prepared by any suitable method known in the art.

For example, an amino acid composition, nutritional composition, supplement, or fortifier may be prepared by blending together a protein source, a carbohydrate source and a fat source in appropriate proportions. If used, emulsifiers may be included at this point. Vitamins and minerals may be added at this point but they may be added later to avoid thermal degradation.

Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The amino acids may be added at this point, especially if the final product is to have a liquid form. If the final product is to be a powder, they may likewise be added at this stage if desired. Alternatively the amino acids may be added later to avoid thermal degradation.

The liquid mixture may then be homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If a liquid composition is preferred, the homogenised mixture may be sterilised then aseptically filled into suitable containers or may be first filled into the containers and then retorted.

If the final product is to be a powder, the homogenised mixture may be transferred to a suitable drying apparatus such as a spray dryer or freeze dryer and converted to powder. The powder may have a moisture content of less than about 5% by weight. The amino acids may also or alternatively be added at this stage by dry-mixing or by blending.

### EXAMPLES

The invention will now be further described by way of examples, which are meant to serve to assist one of ordinary skill in the art in carrying out the invention and are not intended in any way to limit the scope of the invention.

### Example 1—Effect of Amino Acid Blend on Plasma Levels of Arginine and Glutamine in the Critically Ill

### Methods

### Study Design

This proof-of-concept, randomized, double-blind, placebo-controlled trial was performed on two parallel groups. The trial was conducted at an intensive care unit and approved by an independent ethics committee.

Patients were eligible for inclusion if they were aged 18 and over, met criteria for sepsis (Singer M, et al. JAMA 2016; 315 (8): 801-810) or acute respiratory distress syndrome (ARDS) (Ranieri V M, et al. JAMA 2012; 307 (23): 2626-33) within 72 hours of ICU admission, and had an expected length of stay in the ICU or in the intermediate care unit of at least 21 days. Patients were excluded if they exhibited muscle mass loss due to a previous hospitalization, intolerance to enteral feeding, chronic renal failure, chronic liver disease, pacemaker or metal implants incompatible with Magnetic Resonance Imaging (MRI), or if they were cachectic, on long-term parenteral feeding, or pregnant. Patients receiving neuromuscular-blocking agents were also excluded.

Eligible patients with sepsis or ARDS were randomly assigned in a 1:1 ratio to receive a blend of 5 amino acids (threonine, cysteine, proline, serine, and leucine) or their respective placebos. The group receiving the amino acid blend is called “Amino-Acid group” throughout this example. The randomization list was generated by computer and randomization was stratified by age (≤50 years or >50 years) and balanced using dynamic allocation method (Medidata RTSM, second best probability set to 15%). The investigational products and the placebo were in all points similar, presented as powder and were enclosed in identically looking sticks.

Participants, ICU staff, investigators, pharmacists, the statistician and the sponsor remained blinded to the nature of the stick (investigational or placebo) added to the enteral product (Isosource Energy®-Nestle Health Science, France) throughout the study period.

### Study Intervention

Amino acids were administered through the enteral route as a supplement to enteral nutrition. Each stick of active treatment contained 3 g of Threonine, 1.3 g of Proline, 2.5 g of Serine, 2 g of Cysteine, 2.3 g of Leucine, and 10.5 g of maltodextrin. Each dose of investigational product was mixed in a bottle containing 500 ml of Isosource Energy® (Nestlé Health Science, France), accounting for an additional 88 kcal. Isosource Energy® is an enteral product containing 61.0 gram of protein per litre and 1500 kcal per litre. The investigational product was continuously administered over a period of 21 days or until enteral nutrition was interrupted by the physician in charge of the patient. The matching isocaloric placebo containing 22 g maltodextrin was administered following the exact same method of administration as the investigational product.

Patient's management strictly adhered to international guidelines (Rhodes A, et al. Intensive Care Med 2017; 43 (3): 304-377). Briefly, regarding nutrition, full enteral nutrition was initiated as soon as feasible. Nutritional targets ranged between 25-30 kcal/kg/day (McClave S A, et al. JPEN J Parenter Enteral Nutr 2016; 40 (2): 159-211).

### Screening and Follow-Up Visit

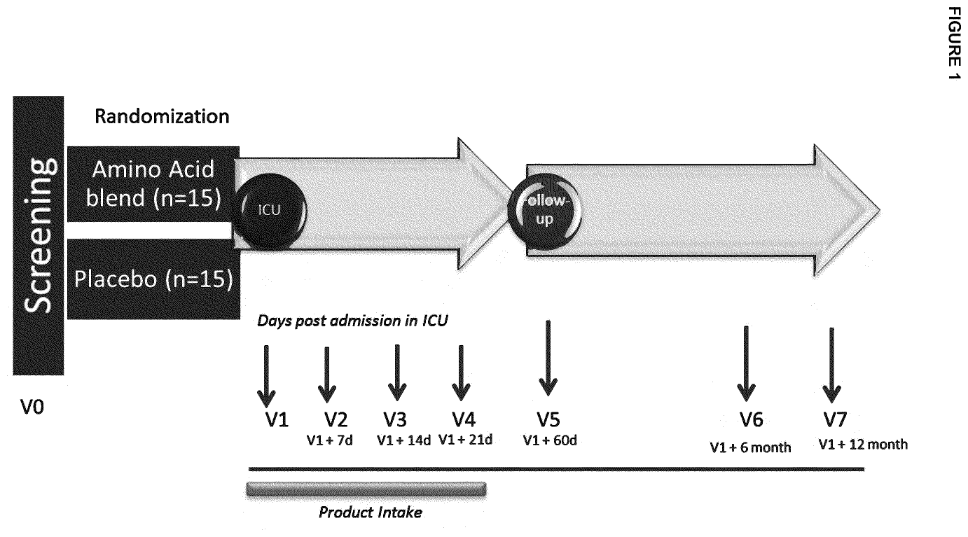

The study design is summarised in FIG. 1. A screening visit occurred upon ICU admission to determine eligibility for the study. Baseline visit (V1) was performed within 72 hours of admission. Follow up visits (V2 to V7) occurred 7, 14, 21, 60, 180 and 365 days after randomisation. During the baseline visit and up to 21 days after randomisation we recorded urine output and serum creatinine.

Functional enterocyte mass was assessed by plasma citrulline concentration (Crenn P, et al. Journal of Critical Care 2014; 29 (2): 315.e1-315.e6).

Diaphragm muscle strength was evaluated by the measurement of twitch mouth pressure in response to magnetic stimulation (Man W D-C. European Respiratory Journal 2004; 24 (5): 846-860; and Windisch W, et al. Chest 2005; 128 (1): 190-195). Briefly, patients were placed in a reclining position and breathed through a flanged mouthpiece or directly through the endotracheal tube and a three way non-re-breathing valve. The mouthpiece or the endotracheal tube was connected to a number 2 Fleisch pneumotachograph (Fleisch, Lausanne, Switzerland) and a differential pressure transducer (MP45±100 cmH2O; Validyne Engineering Corp., Northridge, CA, USA). Twitch mouth pressure was measured after cervical magnetic phrenic nerve stimulation via a 90-mm circular coil powered by a Magstim stimulator. To optimize coil position, several stimulations at 70% of maximal output were performed over the spinal processes at varying levels in the midline between C5 and C7, until optimal stimulation was determined. Through an analogical comparator, the phrenic nerve was stimulated once a predetermined—5 cmH₂O inspiratory pressure trigger was reached (Hua D M, et al. Respiratory Physiology & Neurobiology 2014; 201:47-54). A total of 4 stimulations were performed at maximal stimulator output and the maximal values were recorded for analysis. A 30 seconds delay was programmed between magnetic stimulations in order to prevent potentiation (Wragg S, et al. Thorax 1994; 49 (12): 1234-1237).

Anterior Quadriceps volume measurement was achieved using an isometric T1 weighted 3D with semi-automatic measurement. Following analysis of the overall results, we indexed quadriceps muscle volume to the patient's weight in order to correct for peripheral (muscle) edema.

Plasma amino acids concentrations were measured by ion exchange chromatography with spectrophotometer detection after ninhydrine derivatization (Karagounis L G, et al. Frontiers in Nutrition 2019; 6:181).

### Statistical Analysis

Recruitment proceeded until a sample of 30 patients had completed follow up at day 21. All endpoints were analysed separately without adjusting for multiplicity. Analysis was by intention to treat.

Mean changes from baseline at days 7, 14, 21 and 60 were analysed using a restricted maximum likelihood (REML)-based repeated measures approach. Baseline, treatment group, visit, age and gender were considered as fixed effects while within subject variability was controlled for by considering subject as a random effect. An unstructured covariance structure was used to model the within-patient errors; the covariance structure converging to the best fit, as determined by Akaike's information criterion, was used in the full model. The Kenward-Roger approximation was used to estimate denominator degrees of freedom. Normality and homoscedasticity of variables were checked using the Shapiro-Wilk normality test. Length of stay in the ICU was compared using the Log rank test.

All analyses were conducted with SAS statistical software, version 9.4 (SAS Institute).

### Results

Of 52 patients screened for eligibility, 35 were enrolled, and randomly assigned to receive placebo (n=17) or Amino Acids (n=18). Baseline characteristics of patients are described in Table 1.

TABLE 1
Baseline characteristics

| | Placebo (n = 17) | | Amino Acid (n = 18) | |
|---|---|---|---|---|
| Age (years) | 71 | (62-75) | 71 | (52-83) |
| Gender Male | 12 | (70.6%) | 9 | (50%) |
| Height (cm) | 175 | (163-178) | 165 | (160-175) |
| Weight (kg) | 78.5 | (61.9-91) | 65.1 | (53.5-86.6) |
| BMI | 26.9 | (21.8-28.7) | 24.2 | (22-26) |
| Creatinine (μmol/L) | 73 | (55-130) | 64 | (51-92) |
| Sepsis | 15 | (88) | 18 | (100) |
| ARDS | 2 | (12° | 0 | (0) |
| SOFA | 10 | (8-12) | 9 | (6-11) |
| SAPS II | 50 | (45-60) | 43 | (32-71) |
| Vasopressor support | 5 | (29.4) | 5 | (27.8) |
| Mechanical ventilation | 14 | (82.4) | 17 | (94.4) |

SOFA, sequential organ failure assessment; SAPS II, Simplified Acute Physiology Score Il, BMI body mass index. Data are n/N (%), or median (IQR).

Enteral nutrition was administered for a median of 7.5 [5; 14] days in the Amino Acid arm compared to 10 [7; 22] days in the placebo arm. During the interventional period the total amount of energy provided by enteral nutrition was 10,956 [6769; 25,577] kcal in the experimental arm vs. 14,090 [10,703; 34,554] kcal in the placebo arm (Table 2). Throughout the administration of enteral nutrition, the daily median amount of ingested calories was 1461 [902; 3410] and 1409 [1070; 3455] kcal/day respectively in the Amino Acid and placebo arms.

TABLE 2
Effective amount of enteral nutrition intake by study period
during the interventional period

| | Placebo (n = 17) | | Amino Acid (n = 18) | | P |
|---|---|---|---|---|---|
| Enteral | 10 | (7-22) | 7.5 | (5-14) | 0.3929 |
| nutrition | | | | | |
| administration | | | | | |
| (days) | | | | | |
| Between trial | 11,336 | (7839-13,676) | 8621 | (6769-11,940) | 0.2039 |
| day 1 and | | | | | |
| trial day 7 | | | | | |
| (kcal) | | | | | |
| Between trial | 10,088 | (3007-11,558) | 8522 | (3481-12,490) | 0.8808 |
| day 8 and | | | | | |
| trial day 14 | | | | | |
| (kcal) | | | | | |
| Between trial | 11,522 | (11,085-14,204) | 9126 | (5232-12,542) | 0.3624 |
| day 15 | | | | | |
| and trial | | | | | |
| day 21 (kcal) | | | | | |

Data are median (IQR). Wilcoxon two sample test.

The investigational treatment significantly increased plasma concentration of threonine, proline, serine, cysteine and leucine compared to the placebo arm, on days 7 and 14 after randomisation, demonstrating treatment compliance.

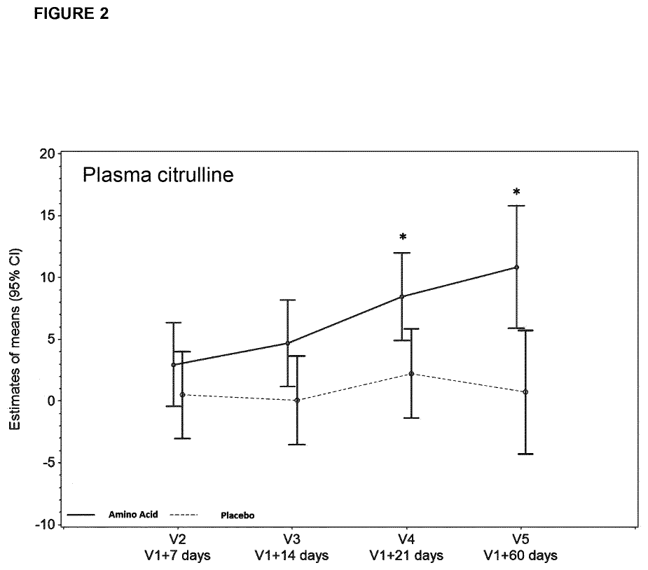

Plasma citrulline, a marker of bioactive enterocyte mass, significantly increased over time (P=0.011 overall) and especially on days 21 (P=0.024) and 60 (P=0.006) in the Amino Acid arm compared to the placebo arm (FIG. 2).

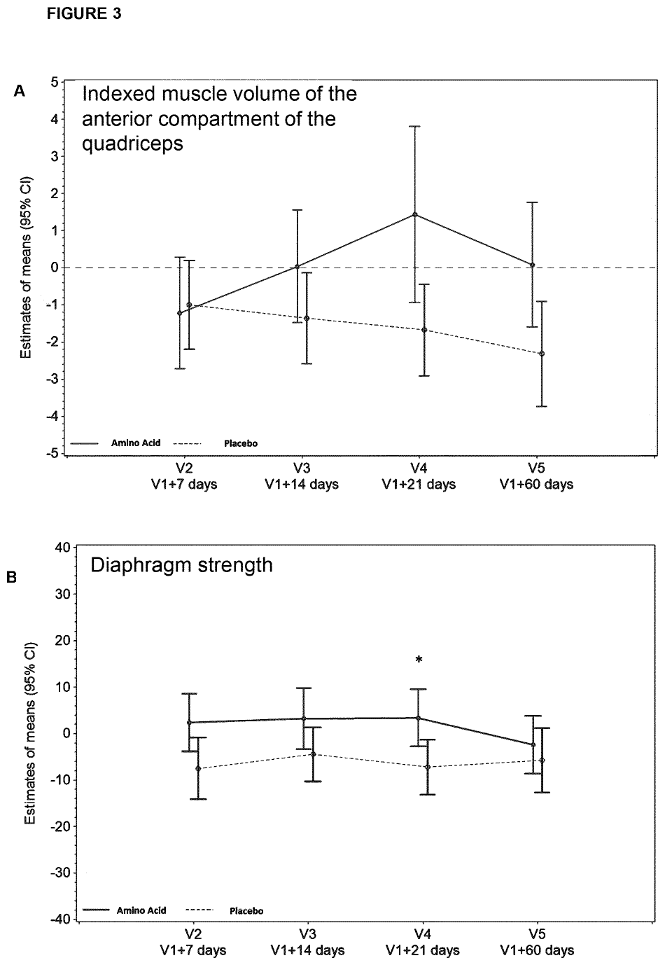

Indexed muscle volume of the anterior compartment of the quadriceps was significantly greater in the Amino Acid arm (P=0.066 overall and P=0.022 on day 21) (FIG. 3A). Diaphragm strength increased from baseline up till 3 weeks after randomization in the Amino Acid group compared to the placebo group. Three weeks after randomization, diaphragm strength was significantly greater in the Amino Acid arm compared to placebo (P=0.035) (FIG. 3B).

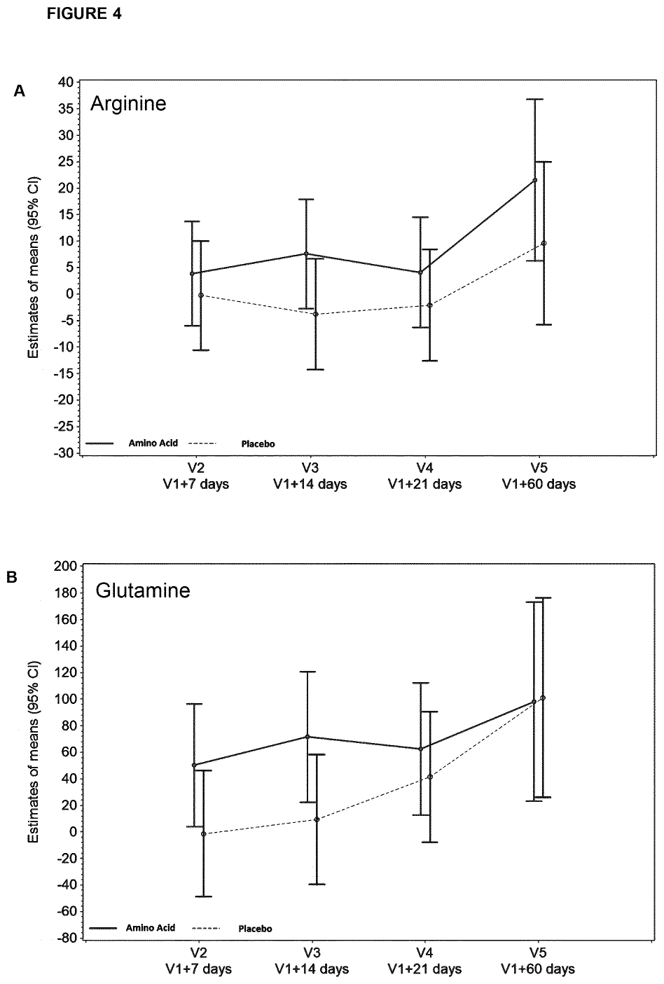

Plasma levels of arginine decreased until visit 3 in the placebo group and plasma levels of glutamine decreased until visit 2 in the placebo group while plasma levels of arginine and glutamine increased over time in the Amino Acid group (FIG. 4).

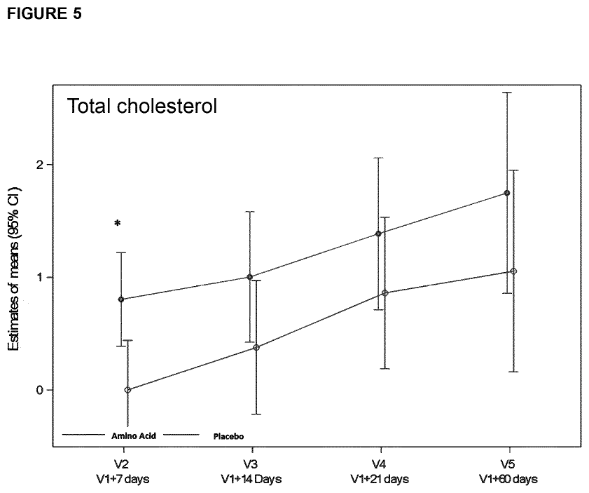

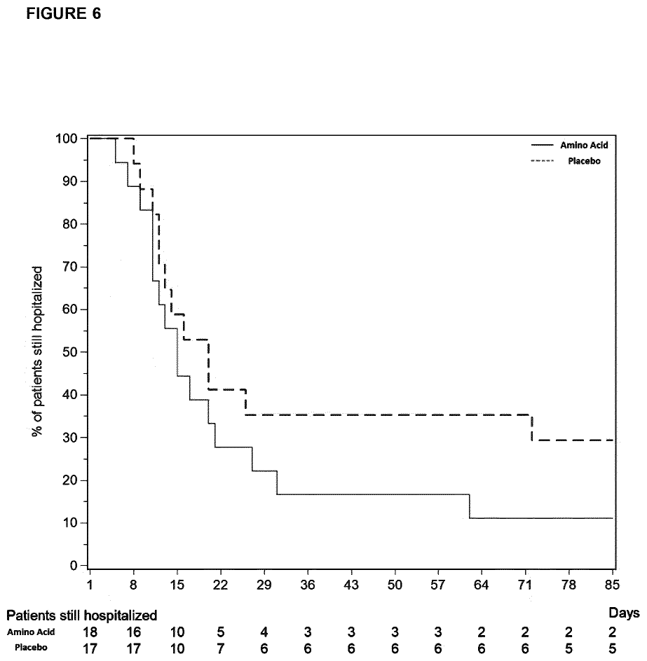

Overall, clinical outcomes were consistently better in patients treated by the investigational product. Cholesterol levels were higher in the interventional arm at one week of treatment (P=0.007) and remained higher throughout the trial (FIG. 5). The median number of ICU free days until day 21 was 9.5 [0; 13] days in the interventional arm vs 6.0 [0; 12] in the placebo arm (P=0.457) (FIG. 6). Ventilation free days until day 21 were 12.5 [4; 18] days in the Amino Acid arm vs 10 [0; 17] days in the placebo arm (P=0.316). 60 days after randomisation, 8/13 (61.5%) patients were walking in the Amino Acid group versus 5/12 (31.7%) in the placebo group (P=0.434).

The experimental product was well tolerated with no evidence of renal impairment based on urine output or serum creatinine (Table 3). Specifically, the administration of Amino Acids was not temporally associated with any evidence of oliguria or of kidney injury. Nine deaths occurred during the trial, 3 deaths in the Amino Acid group vs 6 deaths in the placebo group. No death was related to the administration of the study product.

TABLE 3
Renal function

| Visit | Variable | Placebo N = 17 | | Amino Acid N = 18 | |
|---|---|---|---|---|---|
| V1 (Baseline) | Urine output (mL/24 h) | 1300 | (950-2500) | 1700 | (500-2350) |
| | Serμm creatinine (μmol/L) | 70 | (54-120) | 63 | (51-92) |
| V2 (V1 + 7 days) | Urine output (mL/24 h) | 1300 | (900-2100) | 1050 | (800-2300) |
| | Serμm creatinine (μmol/L) | 72 | (48-94) | 55.5 | (47.5-71) |
| V3 (V1 + 14 days) | Urine output (mL/24 h) | 1375 | (625-2450) | 950 | (725-1975) |
| | Serμm creatinine (μmol/L) | 72 | (51-98) | 57 | (48-92) |
| V4 (V1 + 21 days) | Urine output (mL/24 h) | 1650 | (1000-2000) | 900 | (700-1500) |
| | Serμm creatinine (μmol/L) | 63 | (48-84) | 61 | (37-82) |
| V5 (V1 + 60 days) | Urine output (mL/24 h) | 1800 | (1600-2000) | 900 | (400-5050) |
| | Serμm creatinine (μmol/L) | 52.5 | (51-81) | 66.5 | (50-71) |

Data are median (IQR).

### Conclusion

In this randomized controlled, double blind trial, we showed that enteral supplementation by a specifically tailored blend of threonine, cysteine, proline, serine, and leucine increased citrulline concentration compared to the control group. This indicates that the amino acid blend increased functional enterocyte mass and thus speeds up gut healing and recovery of the gut barrier function.

Critically ill patients and especially long stayers exhibit significant muscle mass loss since the muscles act as role of amino acid reservoirs, providing amino acids to the splanchnic area for anabolism. In this context, it is of particular interest to observe that the amino acid blend increased quadriceps volume and improved diaphragm muscle strength, suggesting that muscle catabolism supply free amino acids is decreased when sufficient amounts of amino acids are supplied through the diet.

An important observation is that plasma levels of glutamine and arginine, both considered conditionally essential amino acids in critical illness did not drop in the supplemented group, suggesting that supplemented amino acids can sustain endogenous synthesis of these amino acids.

We consistently observed trends in favor of a clinical benefit in the Amino Acid group: ventilation free days were 25% higher and patients spent 3 to 4 less days in the ICU in the Amino Acid group. More patients had recovered their ability to walk on day 60 in the Amino Acid group. Moreover, normalisation of cholesterol level is a positive prognostic in ICU patients.

The amino acid blend did not alter renal function measured by creatinine or urine output Moreover, amino acid supplementation significantly increased corresponding amino acid plasma concentration without ever reaching toxic levels. The amino acid blend assessed in the current trial can therefore be considered safe in critically ill patients.

### Embodiments

Various preferred features and embodiments of the present invention will now be described with reference to the following numbered paragraphs (paras).
1. An amino acid composition comprising threonine, serine, proline, leucine, and cysteine for use in preventing and/or reducing glutamine deficiency and/or arginine deficiency in a subject.
2. An amino acid composition comprising threonine, serine, proline, leucine, and cysteine for use in preventing and/or reducing muscle loss in a subject, wherein the subject is at risk of or has a glutamine deficiency and/or an arginine deficiency.
3. The amino acid composition for use according to para 1 or 2, wherein the subject is a critically ill patient.
4. The amino acid composition for use according to any preceding para, wherein the subject has a condition selected from one or more of: sepsis, acute respiratory failure, trauma, surgery, shock, pancreatitis, severe burns, mucositis, inflammatory bowel disease, distorted gut barrier, intestinal mucosal lesions and short-bowel syndrome, and/or the subject is a pre-term infant.
5. The amino acid composition for use according to any preceding para, wherein the amino acid composition improves muscle mass and/or strength, preferably wherein the amino acid composition improves skeletal muscle mass and/or strength, and/or diaphragm muscle strength.
6. The amino acid composition for use according to any preceding para, wherein the amino acid composition promotes gut healing, preferably wherein the amino acid composition promotes recovery of functional enterocyte mass and/or promotes recovery of gut barrier structure and/or function.
7. The amino acid composition for use according to any preceding para, wherein the amino acid composition is administered by enteral administration, preferably wherein the amino acid composition is administered by a feeding tube.
8. The amino acid composition for use according to any preceding para, wherein the amino acid composition is in the form of a nutritional composition, supplement, or fortifier.
9. The amino acid composition for use according to any preceding para, wherein the amino acid composition is in the form of a nutritional composition, preferably wherein the amino acid composition is in the form of an enteral nutritional composition.
10. The amino acid composition for use according to any preceding para, wherein the subject is administered each of threonine, serine, proline, leucine, and cysteine in an amount of about 0.02 g/kg/day or more, about 0.03 g/kg/day or more, or about 0.04 g/kg/day or more.
11. The amino acid composition for use according to any preceding para, wherein the subject is administered each of threonine, serine, proline, leucine, and cysteine in an amount of about 0.5 g/kg/day or less, about 0.4 g/kg/day or less, or about 0.3 g/kg/day or less.
12. The amino acid composition for use according to any preceding para, wherein the subject is administered each of threonine, serine, proline, leucine, and cysteine in an amount of from about 0.02 g/kg/day to about 0.5 g/kg/day, from about 0.03 g/kg/day to about 0.4 g/kg/day, or from about 0.04 g/kg/day to about 0.3 g/kg/day.
13. The amino acid composition for use according to any preceding para, wherein the subject is administered:
   (i) from about 0.06 g/kg/day to about 0.5 g/kg/day, from about 0.08 g/kg/day to about 0.4 g/kg/day, or from about 0.1 g/kg/day to about 0.3 g/kg/day threonine;
   (ii) from about 0.04 g/kg/day to about 0.4 g/kg/day, from about 0.06 g/kg/day to about 0.3 g/kg/day, or from about 0.08 g/kg/day to about 0.2 g/kg/day serine;
   (iii) from about 0.02 g/kg/day to about 0.4 g/kg/day, from about 0.03 g/kg/day to about 0.3 g/kg/day, or from about 0.04 g/kg/day to about 0.2 g/kg/day proline;
   (iv) from about 0.04 g/kg/day to about 0.5 g/kg/day, from about 0.06 g/kg/day to about 0.4 g/kg/day, or from about 0.08 g/kg/day to about 0.3 g/kg/day leucine; and/or
   (v) from about 0.02 g/kg/day to about 0.4 g/kg/day, from about 0.04 g/kg/day to about 0.3 g/kg/day, or from about 0.06 g/kg/day to about 0.2 g/kg/day cysteine.
14. The amino acid composition for use according to any preceding para, wherein the subject is administered each of threonine, serine, proline, leucine, and cysteine in an amount of about 1% or more, about 2% or more, or about 3% or more as a percentage of total protein.
15. The amino acid composition for use according to any preceding para, wherein the subject is administered:
   (i) from about 5% to about 16%, from about 6% to about 14%, or from about 7% to about 12% threonine as a percentage of total protein;
   (ii) from about 4% to about 16%, from about 5% to about 14%, or from about 6% to about 12% serine as a percentage of total protein;
   (iii) from about 1% to about 16%, from about 2% to about 14%, or from about 3% to about 12% proline as a percentage of total protein;
   (iv) from about 3% to about 18%, from about 4% to about 16%, or from about 5% to about 14% leucine as a percentage of total protein; and/or
   (v) from about 2% to about 10%, from about 3% to about 8%, or from about 4% to about 6% cysteine as a percentage of total protein.
16. The amino acid composition for use according to any preceding para, wherein the amino acid composition comprises:
   (i) a weight ratio of threonine:serine of from about 0.5:1 to about 1.5:1;
   (ii) a weight ratio of threonine:proline of from about 0.5:1 to about 2.5:1;
   (iii) a weight ratio of threonine:leucine of from about 0.5:1 to about 1.5:1;
   (iv) a weight ratio of threonine:cysteine of from about 1:1 to about 2.5:1;
   (v) a weight ratio of serine:proline of from about 0.5:1 to about 2:1;
   (vi) a weight ratio of serine:leucine of from about 0.5:1 to about 2:1;
   (vii) a weight ratio of serine:cysteine of from about 1:1 to about 2.5:1;
   (viii) a weight ratio of proline:leucine of from about 0.5:1 to about 1.5:1;
   (ix) a weight ratio of proline:cysteine of from about 0.5:1 to about 2.5:1; and/or
   (x) a weight ratio of leucine:cysteine of from about 1:1 to about 3:1.
17. The amino acid composition for use according to any preceding para, wherein the amino acid composition comprises each of threonine, serine, proline, leucine, and cysteine in an amount of about 0.5 g/1000 kcal or more, about 1 g/1000 kcal or more, or about 1.5 g/1000 kcal or more.
18. The amino acid composition for use according to any preceding para, wherein the amino acid composition comprises:
   (i) from about 1 g/1000 kcal to about 20 g/1000 kcal, from about 2 g/1000 kcal to about 15 g/1000 kcal, or from about 3 g/1000 kcal to about 10 g/1000 kcal threonine;
   (ii) from about 1 g/1000 kcal to about 16 g/1000 kcal, from about 2 g/1000 kcal to about 12 g/1000 kcal, or from about 3 g/1000 kcal to about 8 g/1000 kcal serine;
   (iii) from about 0.5 g/1000 kcal to about 16 g/1000 kcal, from about 1 g/1000 kcal to about 12 g/1000 kcal, or from about 1.5 g/1000 kcal to about 8 g/1000 kcal proline;
   (iv) from about 1 g/1000 kcal to about 20 g/1000 kcal, from about 1.5 g/1000 kcal to about 16 g/1000 kcal, or from about 2 g/1000 kcal to about 12 g/1000 kcal leucine; and/or
   (v) from about 1 g/1000 kcal to about 10 g/1000 kcal, from about 1.5 g/1000 kcal to about 7.5 g/1000 kcal, or from about 2 g/1000 kcal to about 5 g/1000 kcal cysteine.
19. The amino acid composition for use according to any preceding para, wherein the amino acid composition further comprises one or more additional amino acids, preferably wherein the amino acid composition further comprises aspartate, isoleucine, or valine, or any combination thereof.
20. The amino acid composition for use according to any preceding para, wherein the amino acid composition comprises from about 1000 kcal/L to about 2000 kcal/L.
21. The amino acid composition for use according to any preceding para, wherein the amino acid composition comprises protein, preferably wherein the amino acid composition comprises from about 50 g/L to about 120 g/L protein.
22. The amino acid composition for use according to any preceding para, wherein the amino acid composition comprises lipid, preferably wherein the amino acid composition comprises from about 20 g/L to about 100 g/L lipid.
23. The amino acid composition for use according to any preceding para, wherein the amino acid composition comprises carbohydrate, preferably wherein the amino acid composition comprises from about 100 g/L to about 200 g/L carbohydrate.
24. The amino acid composition for use according to any preceding para, wherein the amino acid composition does not comprise added glutamine and/or arginine.
25. An enteral nutritional composition comprising each of threonine, serine, proline, leucine, and cysteine in an amount of about 0.5 g/1000 kcal or more.
26. The enteral nutritional composition according to para 25, wherein the enteral nutritional composition comprises:
   (i) a weight ratio of threonine:serine of from about 0.5:1 to about 1.5:1;
   (ii) a weight ratio of threonine:proline of from about 0.5:1 to about 2.5:1;
   (iii) a weight ratio of threonine:leucine of from about 0.5:1 to about 1.5:1;
   (iv) a weight ratio of threonine:cysteine of from about 1:1 to about 2.5:1;
   (v) a weight ratio of serine:proline of from about 0.5:1 to about 2:1;
   (vi) a weight ratio of serine:leucine of from about 0.5:1 to about 2:1;
   (vii) a weight ratio of serine:cysteine of from about 1:1 to about 2.5:1;
   (viii) a weight ratio of proline:leucine of from about 0.5:1 to about 1.5:1;
   (ix) a weight ratio of proline:cysteine of from about 0.5:1 to about 2.5:1; and/or
   (x) a weight ratio of leucine:cysteine of from about 1:1 to about 3:1.
27. The enteral nutritional composition according to para 25 or 26, wherein the enteral nutritional composition comprises each of threonine, serine, proline, leucine, and cysteine in an amount of about 1 g/1000 kcal or more, or about 1.5 g/1000 kcal or more.
28. The enteral nutritional composition according to any of paras 25 to 27, wherein the enteral nutritional composition comprises each of threonine, serine, proline, leucine, and cysteine in an amount of about 20 g/1000 kcal or less, about 15 g/1000 kcal or less, or about 12 g/1000 kcal or less.
29 The enteral nutritional composition according to any of paras 25 to 28, wherein the enteral nutritional composition comprises each of threonine, serine, proline, leucine, and cysteine in an amount of from about 0.5 g/1000 kcal to about 20 g/1000 kcal, from about 1 g/1000 kcal to about 15 g/1000 kcal, or from about 1.5 g/1000 kcal to about 12 g/1000 kcal.
30. The enteral nutritional composition according to any of paras 25 to 29, wherein the enteral nutritional composition comprises:
   (i) from about 1 g/1000 kcal to about 20 g/1000 kcal, from about 2 g/1000 kcal to about 15 g/1000 kcal, or from about 3 g/1000 kcal to about 10 g/1000 kcal threonine;
   (ii) from about 1 g/1000 kcal to about 16 g/1000 kcal, from about 2 g/1000 kcal to about 12 g/1000 kcal, or from about 3 g/1000 kcal to about 8 g/1000 kcal serine;
   (iii) from about 0.5 g/1000 kcal to about 16 g/1000 kcal, from about 1 g/1000 kcal to about 12 g/1000 kcal, or from about 1.5 g/1000 kcal to about 8 g/1000 kcal proline;
   (iv) from about 1 g/1000 kcal to about 20 g/1000 kcal, from about 1.5 g/1000 kcal to about 16 g/1000 kcal, or from about 2 g/1000 kcal to about 12 g/1000 kcal leucine; and/or
   (v) from about 1 g/1000 kcal to about 10 g/1000 kcal, from about 1.5 g/1000 kcal to about 7.5 g/1000 kcal, or from about 2 g/1000 kcal to about 5 g/1000 kcal cysteine.
31. The enteral nutritional composition according to any of paras 25 to 30, wherein the enteral nutritional composition comprises each of threonine, serine, proline, leucine, and cysteine in an amount of about 1% or more, about 2% or more, or about 3% or more as a percentage of total protein.
32. The enteral nutritional composition according to any of paras 25 to 31, wherein the enteral nutritional composition comprises:
   (i) from about 5% to about 16%, from about 6% to about 14%, or from about 7% to about 12% threonine as a percentage of total protein;
   (ii) from about 4% to about 16%, from about 5% to about 14%, or from about 6% to about 12% serine as a percentage of total protein;
   (iii) from about 1% to about 16%, from about 2% to about 14%, or from about 3% to about 12% proline as a percentage of total protein;
   (iv) from about 3% to about 18%, from about 4% to about 16%, or from about 5% to about 14% leucine as a percentage of total protein; and/or
   (v) from about 2% to about 10%, from about 3% to about 8%, or from about 4% to about 6% cysteine as a percentage of total protein.
33. The enteral nutritional composition according to any of paras 25 to 32, wherein the enteral nutritional composition further comprises one or more additional amino acids, preferably wherein the enteral nutritional composition further comprises aspartate, isoleucine, or valine, or any combination thereof.
34. The enteral nutritional composition according to any of paras 25 to 33, wherein the enteral nutritional composition comprises from about 1000 kcal/L to about 2000 kcal/L.
35. The enteral nutritional composition according to any of paras 25 to 34, wherein the enteral nutritional composition comprises protein, preferably wherein the enteral nutritional composition comprises from about 50 g/L to about 120 g/L protein.
36. The enteral nutritional composition according to any of paras 25 to 35, wherein the enteral nutritional composition comprises lipid, preferably wherein the enteral nutritional composition comprises from about 20 g/L to about 100 g/L lipid.
37. The enteral nutritional composition according to any of paras 25 to 36, wherein the enteral nutritional composition comprises carbohydrate, preferably wherein the enteral nutritional composition comprises from about 50 g/L to about 200 g/L carbohydrate.
38. The enteral nutritional composition according to any of paras 25 to 37, wherein the enteral nutritional composition does not comprise added glutamine and/or arginine.
39. A method of producing an enteral nutritional composition according to any of paras 25 to 38, comprising:
   (i) providing a base enteral nutritional composition; and
   (ii) adding threonine, serine, proline, leucine, and cysteine to the base enteral nutritional composition to provide an enteral nutritional composition according to any of paras 25 to 38.
40. A combination of threonine, serine, proline, leucine, and cysteine for use in preventing and/or reducing glutamine deficiency and/or arginine deficiency in a subject.
41. A combination of threonine, serine, proline, leucine, and cysteine for use in preventing and/or reducing muscle loss in a subject, wherein the subject is at risk of or has a glutamine deficiency and/or an arginine deficiency.

1. A method for use in preventing and/or reducing glutamine deficiency and/or arginine deficiency in a subject comprising administering an amino acid composition comprising threonine, serine, proline, leucine, and cysteine to the subject.

2. A method for use in preventing and/or reducing muscle loss in a subject, wherein the subject is at risk of or has a glutamine deficiency and/or an arginine deficiency comprising administering an amino acid composition comprising threonine, serine, proline, leucine, and cysteine to the subject.

3. Method according to claim 1, wherein the subject is a critically ill patient and has a condition selected from the group consisting of: sepsis, acute respiratory failure, trauma, surgery, shock, pancreatitis, severe burns, mucositis, inflammatory bowel disease, distorted gut barrier, intestinal mucosal lesions, and short-bowel syndrome.

4. Method according to claim 1, wherein the amino acid composition improves muscle mass and/or strength.

5. Method according to claim 1, wherein the amino acid composition is administered by enteral administration.

6. Method according to claim 1, wherein the amino acid composition is in a form selected from the group consisting of a nutritional composition, supplement, and fortifier.

7. Method according to claim 1, wherein the subject is administered each of threonine, serine, proline, leucine, and cysteine in an amount of about 0.02 g/kg/day or more.

8. Method according to claim 1, wherein the subject is administered each of threonine, serine, proline, leucine, and cysteine in an amount of about 1% or more as a percentage of total protein.

9. Method according to claim 1, wherein the amino acid composition comprises each of threonine, serine, proline, leucine, and cysteine in an amount of about 0.5 g/1000 kcal or more.

10. (canceled)

11. Method according to claim 1, wherein the amino acid composition comprises: (i) from about 1 g/1000 kcal to about 20 g/1000 kcal, from about 2 g/1000 kcal to about 15 g/1000 kcal, or from about 3 g/1000 kcal to about 10 g/1000 kcal threonine; (ii) from about 1 g/1000 kcal to about 16 g/1000 kcal, from about 2 g/1000 kcal to about 12 g/1000 kcal, or from about 3 g/1000 kcal to about 8 g/1000 kcal serine; (iii) from about 0.5 g/1000 kcal to about 16 g/1000 kcal, from about 1 g/1000 kcal to about 12 g/1000 kcal, or from about 1.5 g/1000 kcal to about 8 g/1000 kcal proline; (iv) from about 1 g/1000 kcal to about 20 g/1000 kcal, from about 1.5 g/1000 kcal to about 16 g/1000 kcal, or from about 2 g/1000 kcal to about 12 g/1000 kcal leucine; and/or (v) from about 1 g/1000 kcal to about 10 g/1000 kcal, from about 1.5 g/1000 kcal to about 7.5 g/1000 kcal, or from about 2 g/1000 kcal to about 5 g/1000 kcal cysteine.

12. (canceled)

13. Method according to claim 1, wherein the amino acid composition does not comprise added glutamine and/or arginine.

14-15. (canceled)

16. Method according to claim 2, wherein the subject is a critically ill patient and has a condition selected from the group consisting of: sepsis, acute respiratory failure, trauma, surgery, shock, pancreatitis, severe burns, mucositis, inflammatory bowel disease, distorted gut barrier, intestinal mucosal lesions, and short-bowel syndrome.

17. Method according to claim 2, wherein the amino acid composition improves muscle mass and/or strength.

18. Method according to claim 2, wherein the amino acid composition is administered by enteral administration.

19. Method according to claim 2, wherein the amino acid composition is in a form selected from the group consisting of a nutritional composition, supplement, and fortifier.

20. Method according to claim 2, wherein the subject is administered each of threonine, serine, proline, leucine, and cysteine in an amount of about 0.02 g/kg/day or more.

21. Method according to claim 2, wherein the subject is administered each of threonine, serine, proline, leucine, and cysteine in an amount of about 1% or more as a percentage of total protein.

22. Method according to claim 2, wherein the amino acid composition comprises each of threonine, serine, proline, leucine, and cysteine in an amount of about 0.5 g/1000 kcal or more.

23. Method according to claim 2, wherein the amino acid composition comprises: (i) from about 1 g/1000 kcal to about 20 g/1000 kcal, from about 2 g/1000 kcal to about 15 g/1000 kcal, or from about 3 g/1000 kcal to about 10 g/1000 kcal threonine; (ii) from about 1 g/1000 kcal to about 16 g/1000 kcal, from about 2 g/1000 kcal to about 12 g/1000 kcal, or from about 3 g/1000 kcal to about 8 g/1000 kcal serine; (iii) from about 0.5 g/1000 kcal to about 16 g/1000 kcal, from about 1 g/1000 kcal to about 12 g/1000 kcal, or from about 1.5 g/1000 kcal to about 8 g/1000 kcal proline; (iv) from about 1 g/1000 kcal to about 20 g/1000 kcal, from about 1.5 g/1000 kcal to about 16 g/1000 kcal, or from about 2 g/1000 kcal to about 12 g/1000 kcal leucine; and/or (v) from about 1 g/1000 kcal to about 10 g/1000 kcal, from about 1.5 g/1000 kcal to about 7.5 g/1000 kcal, or from about 2 g/1000 kcal to about 5 g/1000 kcal cysteine.
